SEQUENCE LISTING (30 sequences)

SEQ ID NO: 1 - DNA, 25 bp, Artificial Sequence
  Synthetic oligonucleotide
ctagctagcatgaagaaccatttgc

SEQ ID NO: 2 - DNA, 26 bp, Artificial Sequence
  Synthetic oligonucleotide
tgcgatatcttagtcaggatagcagg

SEQ ID NO: 3 - DNA, 27 bp, Artificial Sequence
  Synthetic oligonucleotide
ctagctagccgccaccatggagcttgg

SEQ ID NO: 4 - DNA, 27 bp, Artificial Sequence
  Synthetic oligonucleotide
cccttgaagcttgctgaagagacggtg

SEQ ID NO: 5 - DNA, 28 bp, Artificial Sequence
  Synthetic oligonucleotide
ctagctagccgccaccatggacatgagg

SEQ ID NO: 6 - DNA, 34 bp, Artificial Sequence
  Synthetic oligonucleotide
gacagatggtgcggccgcagttcgnttgatatcc

SEQ ID NO: 7 - DNA, 25 bp, Artificial Sequence
  Synthetic oligonucleotide
ctagctagcatgaagaaccatttgc

SEQ ID NO: 8 - DNA, 26 bp, Artificial Sequence
  Synthetic oligonucleotide
tgcgatatcttagtcaggatagcagg

SEQ ID NO: 9 - protein, 123 aa, Artificial Sequence
  Synthetic polypeptide
QEQLVESGGGVVQPGRSLRLSCEASGFIFSAFVLHWVRQAPGKGLEWVAVVWYDGNSKYYADSVKGRFTISRDTSQNTLHLQMDSLRPEDTAVYYCAREWVADDDTFDGFDVWGQGTMVTVSS

SEQ ID NO: 10 - DNA, 369 bp, Artificial Sequence
  Synthetic oligonucleotide
caggagcagctggtggagtctgggggaggagtggtccagcctgggaggtccctgagactctcctgtgaagcgtctggtttcatcttcagtgcctttgtcttgcactgggtccgacaggctccaggcaaggggctggagtgggtggcagttgtttggtatgatggaaatagtaaatactatgcagactccgtgaagggccgattcaccatctccagagacacttcccagaacacactgcatctgcaaatggacagcctgcgtcccgaggacacggctgtctattactgtgcgagagaatgggtggcggacgatgatacttttgatggttttgatgtctggggccaagggacaatggtcaccgtctcttca

SEQ ID NO: 11 - protein, 353 aa, Artificial Sequence
  Synthetic polypeptide
ASLTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPDRDLCGCYSVSSVLSGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 12 - DNA, 1059 bp, Artificial Sequence
  Synthetic oligonucleotide
gcaagcttgaccagccccaaggtcttcccgctgagcctctgcagcacccagccagatgggaacgtggtcatcgcctgcctggtccagggcttcttcccccaggagccactcagtgtgacctggagcgaaagcggacagggcgtgaccgccagaaacttcccacccagccaggatgcctccggggacctgtacaccacgagcagccagctgaccctgccggccacacagtgcctagccggcaagtccgtgacatgccacgtgaagcactacacgaatcccagccaggatgtgactgtgccctgcccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgaccgtgacctctgtggctgctacagcgtgtccagtgtcctgtcgggctgtgccgagccatggaaccatgggaagaccttcacttgcactgctgcctaccccgagtccaagaccccgctaaccgccaccctctcaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctac

SEQ ID NO: 13 - protein, 110 aa, Artificial Sequence
  Synthetic polypeptide
EIVLSQSPATLSLSPGERATLSCRASQSVTNSLAWYQQKPGQAPRLLIYDASNRATGIPARFSGSGSGTDFTLTISSLEPEDFAVYYCQQRSNWPPEVTFGPGTKVDIKR

SEQ ID NO: 14 - DNA, 330 bp, Artificial Sequence
  Synthetic oligonucleotide
gaaattgtgttgtcacagtctccagccaccctgtctttgtctccaggggaaagagccaccctctcctgcagggccagtcagagtgttaccaactccttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggaggtcactttcggccctgggaccaaagtggatatcaaacga

SEQ ID NO: 15 - protein, 106 aa, Artificial Sequence
  Synthetic polypeptide
TAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 318 bp, Artificial Sequence
  Synthetic oligonucleotide
actgcggccgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 17 - protein, 8 aa, Artificial Sequence
  Synthetic polypeptide
GFIFSAFV

SEQ ID NO: 18 - protein, 8 aa, Artificial Sequence
  Synthetic polypeptide
VWYDGNSK

SEQ ID NO: 19 - protein, 16 aa, Artificial Sequence
  Synthetic polypeptide
AREWVADDDTFDGFDV

SEQ ID NO: 20 - DNA, 24 bp, Artificial Sequence
  Synthetic oligonucleotide
ggtttcatcttcagtgcctttgtc

SEQ ID NO: 21 - DNA, 24 bp, Artificial Sequence
  Synthetic oligonucleotide
gtttggtatgatggaaatagtaaa

SEQ ID NO: 22 - DNA, 48 bp, Artificial Sequence
  Synthetic oligonucleotide
gcgagagaatgggtggcggacgatgatacttttgatggttttgatgtc

SEQ ID NO: 23 - protein, 6 aa, Artificial Sequence
  Synthetic polypeptide
QSVTNS

SEQ ID NO: 24 - protein, 3 aa, Artificial Sequence
  Synthetic polypeptide
DAS

SEQ ID NO: 25 - protein, 11 aa, Artificial Sequence
  Synthetic polypeptide
QQRSNWPPEVT

SEQ ID NO: 26 - DNA, 18 bp, Artificial Sequence
  Synthetic oligonucleotide
cagagtgttaccaactcc

SEQ ID NO: 27 - DNA, 9 bp, Artificial Sequence
  Synthetic oligonucleotide
gatgcatcc

SEQ ID NO: 28 - DNA, 33 bp, Artificial Sequence
  Synthetic oligonucleotide
cagcagcgtagcaactggcctccggaggtcact

SEQ ID NO: 29 - protein, 486 aa, Artificial Sequence
  Synthetic polypeptide
MEFGLSWVFLVAIIIGVQCQVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGQGTTVIVSSASLTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRLAGKPTHINVSVVMAEADGTCY

SEQ ID NO: 30 - protein, 236 aa, Artificial Sequence
  Synthetic polypeptide
MEFGLSWVFLVAIIIGVQCQVEIVLTQAPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKRTAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2016/0039913 A1
(43) Pub. Date: Feb. 11, 2016

(54) THE USE OF ANTIBODIES IN TREATING HIV INFECTION AND SUPPRESSING HIV TRANSMISSION

(71) Applicants: LFB USA, INC., Framingham, MA (US); BETH ISRAEL DEACONESS MEDICAL CENTER, INC., Boston, MA (US)

(72) Inventors: Harry M. MEADE, Newton, MA (US); Lisa CAVACINI, Natick, MA (US)

(21) Appl. No.: 14/427,117

(22) PCT Filed: Sep. 10, 2013
(86) PCT No.: PCT/US2013/058929

§ 371 Date: Mar. 10, 2015

Related U.S. Application Data
(60) Provisional application No. 61/698,826, filed on Sep. 10, 2012.

(51) Int. Cl. C07K 16/10

(52) U.S. Cl. CPC C07K 16/1045

(57) ABSTRACT

In one aspect, the disclosure provides methods of treating HIV and decreasing the chance of HIV infection in a subject, and compositions used in these methods.

### RELATED APPLICATIONS

This application claims the benefit under 35 U.S.C. §119 of U.S. provisional application 61/698,826 filed Sep. 10, 2012, the entire contents of which are incorporated herein by reference.

### STATEMENT REGARDING FEDERALLY SPONSORED RESEARCH OR DEVELOPMENT

This invention was made with government support under grant numbers AI063986, AI075932 and AI106478 awarded by the NIH. The government has certain rights in the invention.

### FIELD OF THE INVENTION

The disclosure relates to methods of treating HIV infection and suppressing HIV transmission.

### BACKGROUND OF THE INVENTION

A variety of therapies are available to treat HIV infection or decrease the chance of HIV infection upon exposure to the virus. However, there is still no cure or effective vaccine for HIV infection. New methods of treating HIV infection and methods to decrease the chance of HIV infection are needed therefore.

### SUMMARY OF THE INVENTION

In one aspect the disclosure provides methods of, and compositions for, treating HIV and decreasing the chance of HIV infection in a subject.

In one aspect the disclosure provides a method of treating HIV infection in a subject, the method comprising administering to the subject a composition comprising IgA antibody to treat HIV infection. In some embodiments, the composition further comprises milk. In some embodiments, the IgA antibody is a milk-produced IgA antibody. In some embodiments, the milk-produced IgA antibody is produced in the mammary gland of a transgenic non-human mammal. In some embodiments, the milk is produced in the mammary gland of a transgenic non-human mammal that produces IgA antibody in its mammary gland.

In one aspect the disclosure provides a method of treating HIV infection in a subject, the method comprising administering to the subject a composition comprising multimeric antibody to treat HIV infection. In some embodiments, the composition further comprises milk. In some embodiments, the multimeric antibody is a milk-produced multimeric antibody. In some embodiments, the milk-produced multimeric antibody is produced in the mammary gland of a transgenic non-human mammal. In some embodiments, the milk is produced in the mammary gland of a transgenic non-human mammal that produces multimeric antibody in its mammary gland.

In one aspect the disclosure provides a method of treating HIV infection in a subject, the method comprising administering to the subject a composition comprising highly glycosylated antibody to treat HIV infection. In some embodiments, the composition further comprises milk. In some embodiments, the highly glycosylated antibody is a milk-produced highly glycosylated antibody. In some embodiments, the milk-produced highly glycosylated antibody is produced in the mammary gland of a transgenic non-human mammal. In some embodiments, the milk is produced in the mammary gland of a transgenic non-human mammal that produces highly glycosylated antibody in its mammary gland.

In one aspect the disclosure provides a method of decreasing the chance of HIV infection in a subject, the method comprising administering to the subject a composition comprising IgA antibody to decrease the chance of HIV infection. In some embodiments, the composition further comprises milk. In some embodiments, the IgA antibody is a milk-produced IgA antibody. In some embodiments, the milk-produced IgA antibody is produced in the mammary gland of a transgenic non-human mammal. In some embodiments, the milk is produced in the mammary gland of a transgenic non-human mammal that produces IgA antibody in its mammary gland.

In one aspect the disclosure provides a method of decreasing the chance of HIV infection in a subject, the method comprising administering to the subject a composition comprising multimeric antibody to decrease the chance of HIV infection. In some embodiments, the composition further comprises milk. In some embodiments, the multimeric antibody is a milk-produced multimeric antibody. In some embodiments, the milk-produced multimeric antibody is produced in the mammary gland of a transgenic non-human mammal. In some embodiments, the milk is produced in the mammary gland of a transgenic non-human mammal that produces multimeric antibody in its mammary gland.

In one aspect the disclosure provides a method of decreasing the chance of HIV infection in a subject, the method comprising administering to the subject a composition comprising highly glycosylated antibody to decrease the chance of HIV infection. In some embodiments, the composition further comprises milk. In some embodiments, the highly glycosylated antibody is a milk-produced highly glycosylated antibody. In some embodiments, the milk-produced highly glycosylated antibody is produced in the mammary gland of a transgenic non-human mammal. In some embodiments, the milk is produced in the mammary gland of a transgenic non-human mammal that produces highly glycosylated antibody in its mammary gland.

In one aspect the disclosure provides a method of decreasing the chance of HIV infection in a subject that receives breast milk, the method comprising administering to breast milk a composition comprising IgA antibody to decrease the chance of HIV infection in a subject that receives the breast milk. In some embodiments, the composition further comprises milk. In some embodiments, the IgA antibody is a milk-produced IgA antibody. In some embodiments, the milk-produced IgA antibody is produced in the mammary gland of a transgenic non-human mammal. In some embodiments, the milk is produced in the mammary gland of a transgenic non-human mammal that produces IgA antibody in its mammary gland.

In one aspect the disclosure provides a method of decreasing the chance of HIV infection in a subject that receives breast milk, the method comprising administering to breast milk a composition comprising multimeric antibody to decrease the chance of HIV infection in a subject that receives the breast milk. In some embodiments, the composition further comprises milk. In some embodiments, the multimeric antibody is a milk-produced multimeric antibody. In some embodiments, the milk-produced multimeric antibody is produced in the mammary gland of a transgenic non-human mammal. In some embodiments, the milk is produced in the mammary gland of a transgenic non-human mammal that produces multimeric antibody in its mammary gland.

In one aspect the disclosure provides a method of decreasing the chance of HIV infection in a subject that receives breast milk, the method comprising administering to breast milk a composition comprising highly glycosylated antibody to decrease the chance of HIV infection in a subject that receives the breast milk. In some embodiments, the composition further comprises milk. In some embodiments, the highly glycosylated antibody is a milk-produced highly glycosylated antibody. In some embodiments, the milk-produced highly glycosylated antibody is produced in the mammary gland of a transgenic non-human mammal. In some embodiments, the milk is produced in the mammary gland of a transgenic non-human mammal that produces highly glycosylated antibody in its mammary gland.

In one aspect the disclosure provides a method of suppressing mother-to-child transmission of HIV, the method comprising applying a composition comprising IgA antibody to the nipple of the mother prior to breast feeding to suppress mother-to-child transmission of HIV. In some embodiments, the mother is not a wet nurse. In some embodiments, the composition further comprises milk. In some embodiments, the IgA antibody is a milk-produced IgA antibody. In some embodiments, the milk-produced IgA antibody is produced in the mammary gland of a transgenic non-human mammal. In some embodiments, the milk is produced in the mammary gland of a transgenic non-human mammal that produces IgA antibody in its mammary gland.

In one aspect the disclosure provides a method of suppressing mother-to-child transmission of HIV, the method comprising applying a composition comprising multimeric antibody to the nipple of the mother prior to breast feeding to suppress mother-to-child transmission of HIV. In some embodiments, the mother is not a wet nurse. In some embodiments, the composition further comprises milk. In some embodiments, the multimeric antibody is a milk-produced multimeric antibody. In some embodiments, the milk-produced multimeric antibody is produced in the mammary gland of a transgenic non-human mammal. In some embodiments, the milk is produced in the mammary gland of a transgenic non-human mammal that produces multimeric antibody in its mammary gland.

In one aspect the disclosure provides a method of suppressing mother-to-child transmission of HIV, the method comprising applying a composition comprising highly glycosylated antibody to the nipple of the mother prior to breast feeding to suppress mother-to-child transmission of HIV. In some embodiments, the mother is not a wet nurse. In some embodiments, the composition further comprises milk. In some embodiments, the highly glycosylated antibody is a milk-produced highly glycosylated antibody. In some embodiments, the milk-produced highly glycosylated antibody is produced in the mammary gland of a transgenic non-human mammal. In some embodiments, the milk is produced in the mammary gland of a transgenic non-human mammal that produces highly glycosylated antibody in its mammary gland.

In one aspect the disclosure provides a method of suppressing mother-to-child transmission of HIV, the method comprising expressing IgA antibody in one or more cells of the mammary gland of the mother to suppress mother-to-child transmission of HIV. In some embodiments, the mother is not a wet nurse. In some embodiments, the composition further comprises milk. In some embodiments, the IgA antibody is a milk-produced IgA antibody. In some embodiments, the milk-produced IgA antibody is produced in the mammary gland of a transgenic non-human mammal. In some embodiments, the milk is produced in the mammary gland of a transgenic non-human mammal that produces IgA antibody in its mammary gland.

In one aspect the disclosure provides a method of suppressing mother-to-child transmission of HIV, the method comprising administering to the mother a composition comprising IgA antibody to suppress mother-to-child transmission of HIV. In some embodiments, the mother is not a wet nurse. In some embodiments, the composition further comprises milk. In some embodiments, the IgA antibody is a milk-produced IgA antibody. In some embodiments, the milk-produced IgA antibody is produced in the mammary gland of a transgenic non-human mammal. In some embodiments, the milk is produced in the mammary gland of a transgenic non-human mammal that produces IgA antibody in its mammary gland.

In one aspect the disclosure provides a method of decreasing the chance of HIV infection in a subject, the method comprising applying to a mucous membrane of the subject a composition comprising IgA antibody thereby decreasing the chance of HIV infection in the subject. In some embodiments, the composition is applied in a vaginal crème. In some embodiments, the composition further comprises milk. In some embodiments, the IgA antibody is a milk-produced IgA antibody. In some embodiments, the milk-produced IgA antibody is produced in the mammary gland of a transgenic non-human mammal. In some embodiments, the milk is produced in the mammary gland of a transgenic non-human mammal that produces IgA antibody in its mammary gland.

In one aspect the disclosure provides a method of decreasing the chance of HIV infection in a subject, the method comprising applying to a mucous membrane of the subject a composition comprising multimeric antibody thereby decreasing the chance of HIV infection in the subject. In some embodiments, the composition is applied in a vaginal crème. In some embodiments, the composition further comprises milk. In some embodiments, the multimeric antibody is a milk-produced multimeric antibody. In some embodiments, the milk-produced multimeric antibody is produced in the mammary gland of a transgenic non-human mammal. In some embodiments, the milk is produced in the mammary gland of a transgenic non-human mammal that produces multimeric antibody in its mammary gland.

In one aspect the disclosure provides a method of decreasing the chance of HIV infection in a subject, the method comprising applying to a mucous membrane of the subject a composition comprising highly glycosylated antibody thereby decreasing the chance of HIV infection in the subject. In some embodiments, the composition is applied in a vaginal crème. In some embodiments, the mother is not a wet nurse. In some embodiments, the composition further comprises milk. In some embodiments, the highly glycosylated antibody is a milk-produced highly glycosylated antibody. In some embodiments, the milk-produced highly glycosylated antibody is produced in the mammary gland of a transgenic non-human mammal. In some embodiments, the milk is produced in the mammary gland of a transgenic non-human mammal that produces highly glycosylated antibody in its mammary gland.

In any of the methods described herein, the administration or application results in the suppression of HIV replication in a target cell population. In any of the methods described herein, the composition is capable of inducing antibody-dependent cell-mediated viral inhibition (ADCVI). In any of the methods described herein, the method further comprises administering anti-retroviral therapy.

In one aspect the disclosure provides a composition comprising milk-produced IgA antibody. In some embodiments, the milk-produced IgA antibody is produced in the mammary gland of a transgenic non-human mammal. In some embodiments, the composition further comprises milk. In some embodiments, the milk is produced in the mammary gland of a transgenic non-human mammal that produces IgA antibody in its mammary gland. In some embodiments, the composition further comprises a pharmaceutically acceptable carrier.

In one aspect the disclosure provides a composition comprising milk-produced multimeric antibody. In some embodiments, the milk-produced multimeric antibody is produced in the mammary gland of a transgenic non-human mammal. In some embodiments, the composition further comprises milk. In some embodiments, the milk is produced in the mammary gland of a transgenic non-human mammal that produces multimeric antibody in its mammary gland. In some embodiments, the composition further comprises a pharmaceutically acceptable carrier.

In one aspect the disclosure provides a composition comprising milk-produced highly glycosylated antibody. In some embodiments, the milk-produced highly glycosylated antibody is produced in the mammary gland of a transgenic non-human mammal. In some embodiments, the composition further comprises milk. In some embodiments, the milk is produced in the mammary gland of a transgenic non-human mammal that produces highly glycosylated antibody in its mammary gland. In some embodiments, the composition further comprises a pharmaceutically acceptable carrier.

In any of the methods or compositions described herein, in some embodiments, the IgA antibody is an IgA1 antibody.

In any of the methods or compositions described herein, in some embodiments, the IgA antibody is a dimeric IgA1 antibody.

In any of the methods or compositions described herein, in some embodiments, the IgA antibody is an IgA2 antibody.

In any of the methods or compositions described herein, in some embodiments, the IgA antibody binds gp120.

In any of the methods or compositions described herein, in some embodiments, the IgA antibody binds the CD4 binding site on gp120.

In any of the methods or compositions described herein, in some embodiments, the IgA antibody is a b12 antibody.

In any of the methods or compositions described herein, in some embodiments, the IgA antibody is a b12 IgA2 antibody.

In any of the methods or compositions described herein, in some embodiments, the IgA antibody comprises a heavy chain having SEQ ID NO:29.

In any of the methods or compositions described herein, in some embodiments, the IgA antibody comprises a light chain having SEQ ID NO:30.

In any of the methods or compositions described herein, in some embodiments, the IgA antibody the comprises a heavy chain having SEQ ID NO:29 and a light chain having SEQ ID NO:30.

In any of the methods or compositions described herein, in some embodiments, the IgA antibody comprises CDR3: AREWVADDDTFDGFDV (SEQ ID NO:19).

In any of the methods or compositions described herein, in some embodiments, the IgA antibody comprises CDR1: GFIFSAFV (SEQ ID NO:17), CDR2: VWYDGNSK (SEQ ID NO:18), and CDR3: AREWVADDDTFDGFDV (SEQ ID NO:19).

In any of the methods or compositions described herein, in some embodiments, the IgA antibody comprises CDR3: QQRSNWPPEVT (SEQ ID NO:25).

In any of the methods or compositions described herein, in some embodiments, the IgA antibody comprises CDR1: QSVTNS (SEQ ID NO:23), CDR2: DAS (SEQ ID NO:24) and CDR3: QQRSNWPPEVT (SEQ ID NO:25).

In any of the methods or compositions described herein, in some embodiments, the IgA antibody is a F425-A1g8 antibody.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the Figures. The invention is capable of other embodiments and of being practiced or of being carried out in various ways. Also, the phraseology and terminology used herein is for the purpose of description and should not be regarded as limiting.

### BRIEF DESCRIPTION OF THE DRAWINGS

The figures are illustrative only and are not required for enablement of the disclosure.

FIG. 1 shows the identification of b12A2 antibody secreted in the milk of transgenic mice using western blot: Lanes 1-8 are milk samples collected from individual mice; lane 9 is the molecular weight marker; lane 10 is milk from a negative control mouse; lane 11 is purified human IgA; lane 12 is negative control mouse milk spiked with purified human IgA. Reactive bands were detected using biotinylated goat anti-human IgA followed by streptavidin HRP.

FIG. 2 shows the neutralization of HIV (67970) by b12 IgA2 Variants: Cell-free HIV was incubated with serial dilutions of control b12 IgG1 antibody (black diamond), b12 IgA2 purified from CHO cells (grey triangle) and b12 IgA2 expressed in milk (black square) prior to the addition of TZM-b1 cells. HIV was measured as b-galactosidase activity after 48 hours. Percent neutralization was determined by the formula ((control−test)/control)*100.

FIG. 3 shows the immunoreactivity of F425A1g8 IgG1 and IgA1 variants with HIV-infected cells. SF-2 infected cells (1×10⁶) were incubated with titered Abs of F425A1g8 (black squares) and IgA1 (black triangles), which were detected using HRP-conjugated goat anti-human IgG or IgA. Bound Ab was visualized using tetramethylbenzidine substrate and stopped by 100 microliters of 1M phosphoric acid. The OD was read on a plate reader at 450 nm. b12 IgG1 or IgA1 (20 micorgram/ml) was a standard to determine relative activity of the F425A1g8 variants with HIV.

FIG. 4 shows the neutralization activity of F425-A1g8 IgG1 and IgA1 antibody variants against HIV (JR-FL) measured using TZM-b1 cells. JR-FL (100 TCID₅₀) was incubated with two-fold serial dilutions of F425A1g8 IgG1 (open diamond) and IgA1 (black square) variants prior to addition of TZM-b1 cells. HIV was measured as beta-galactosidase activity after 48 h. Percent neutralization was determined by the formula [(control-test)/control]×100.

FIG. 5 shows antibody-dependent cell mediated viral inhibition (ADCVI) mediated by F425-A18 IgA1 and IgG antibody variants measured using HIV (JR-FL) infected peripheral blood mononuclear cells (PBMC). ADCVI mediated by F425A1g8 IgG1 (grey squares) and IgA1 (black diamonds) antibodies and neutrophils. F425A1g8 variants were incubated with JR-FL-infected PBMCs just prior to adding neutrophils at an E:T ratio of 10:1. After 4 h, PHA-stimulated PBMCs were added as indicator cells, and p24 was quantitated by ELISA after 1 wk. Percent inhibition was determined by the following formula: [(p24 control−p24 test)/p24 control]×100.

### DETAILED DESCRIPTION OF THE INVENTION

In one aspect the disclosure provides methods of, and compositions for, treating HIV infection in a subject, decreasing the chance of HIV infection in a subject, decreasing the chance of HIV infection in a subject that receives breast milk, and suppressing mother-to-child transmission of HIV. In some embodiments, the methods and compositions disclosed herein include IgA antibody, highly glycosylated antibody and/or multimeric antibody. In some embodiments, the antibody (e.g., IgA antibody) is a milk-produced antibody. In some embodiments, the milk-produced antibody (e.g., IgA antibody) is produced in the mammary gland of a transgenic mammal. In some embodiments, the composition further comprises milk. In some embodiments, the milk is produced in the mammary gland of a transgenic non-human mammal that produces the antibody (e.g., IgA antibody) in its mammary gland.

As provided herein, milk-produced antibodies (e.g., IgA antibodies) are more effective in methods of treating HIV infection and decreasing the chance of HIV infection than antibodies that were not milk-produced. In addition, it is shown herein that milk-produced antibodies (e.g., IgA antibodies) in combination with milk show a synergistic effect in the treatment of HIV infection and decreasing the chance of HIV infection.

### IgA Antibodies

In some embodiments, the methods of treating HIV disclosed herein comprise administering to the subject a composition comprising IgA antibody to treat HIV infection. In some embodiments, the methods of decreasing the chance of HIV infection in a subject comprise administering to the subject a composition comprising IgA antibody to decrease the chance of HIV infection. In some embodiments, the methods of decreasing the chance of HIV infection in a subject that receives breast milk comprise administering to breast milk a composition comprising IgA antibody to decrease the chance of HIV infection in a subject that receives the breast milk. In some embodiments, the methods of suppressing mother-to-child transmission of HIV comprise applying a composition comprising IgA antibody to the nipple of the mother prior to breast feeding to suppress mother-to-child transmission of HIV. In some embodiments, the methods of suppressing mother-to-child transmission of HIV comprise expressing IgA antibody in one or more cells of the mammary gland of the mother to suppress mother-to-child transmission of HIV. In some embodiments, the methods of suppressing mother-to-child transmission of HIV comprise administering to the mother a composition comprising IgA antibody to suppress mother-to-child transmission of HIV. In some embodiments, the methods of decreasing the chance of HIV infection in a subject comprise applying to a mucous membrane of the subject a composition comprising IgA antibody thereby decreasing the chance of HIV infection in the subject.

In one aspect the disclosure provides methods that include the use of compositions comprising IgA antibodies. Immunoglobulin A (IgA) is an antibody that plays a critical role in mucosal immunity. More IgA is produced in mucosal linings than all other types of antibody combined (Approximately 75% of the total immunoglobulin produced in the entire body). IgA is a first line of defense in maintenance the integrity our mucosa, the immune system manufactures and secretes dimeric IgA to neutralize pathogenic organisms and exclude the entry of commensals at the mucosal border. Dimerized IgA antibodies include a J chain, which facilitates production and dimerization of the IgA antibody.

IgA exists in two isotypes, IgA1 and IgA2, with IgA1 predominating in serum. In IgA2, the heavy and light chains are not linked with disulfide, but with non-covalent bonds. In secretory lymphoid tissues (e.g., gut associated lymphoid tissue, GALT), the share of IgA2 production is larger than in the non-secretory lymphoid organs (e.g., spleen, peripheral lymph nodes). Both IgA1 and IgA2 have been found in external secretions like colostrum, maternal milk, tears and saliva, where IgA2 is more prominent than in the blood.

In the harsh mucosal environment, glycosylated residues help protect the protein from proteases. In comparison to IgG, which is only 2.9% (w/w) glycosylated, IgA1 is 9.5% (w/w) and IgA2 is 11% (w/w) glycosylated. Both IgA1 and IgA2 display N-glycosylated residues. IgA1 has three N-glycosylated residues, on beta strand B on the Ch2 chain and on the J tail. In IgA2, additional sites of N-glycosylation include an Asn on the beta strand G of Ch1 and an Asn of beta strand G on Ch2. Some alloforms of IgA2 are further glycosylated at an additional Asn211 on Ch2. An increased need for protection against proteolytic cleavage at the hinge region accounts for the presence of O-glycosylation in IgA1's hinge region, particularly cleavage by bacterial metalloproteases. The glycosylation residues provide increased steric hindrance, and creating difficulty in fitting the peptide in the protease's active site.

It should be appreciated that antibodies can be class-switched. Thus, for instance, IgG antibodies can be class-switched to become IgA antibodies, including IgA1 and IgA2 (or IgM antibodies). Antibodies directed against a particular antigen are often developed as IgG isotype and subsequently class-switched, such as, for instance, the anti-HIV b12 antibody described herein (See also, Mantis et al. 2007, J. Immunol. 179:3144-3152). Additional anti-HIV antibodies that have been class-switched include 2F5 and 2G12, which have been class-switched from IgG1 to IgM and IgA1 (Wolbank et al. 2003, J. Virol. 77: 4095-4103). A further anti-HIV antibody that has been class-switched is the F425A1g8 antibody (Yu et al. Journal of Immunology 2013, 190: 205-210)

In some embodiments, the IgA antibody is a milk-produced antibody. In some embodiments, the IgA antibody is produced in the mammary gland of a transgenic non-human mammal. In some embodiments, the IgA antibody is a dimeric IgA1 antibody. In some embodiments, the IgA antibody is an IgA2 antibody.

In some embodiments, the IgA antibody has a glycosylation pattern (i.e., the nature and structure of the glycosylation side chain) that is associated with a milk-produced antibody. In some embodiments, the IgA antibody has a glycosylation pattern that is associated with antibodies produced in the mammary gland of a transgenic non-human mammal.

In some embodiments, the IgA antibody is a b12 antibody. In some embodiments, the IgA antibody is a b12 IgA2 antibody. In some embodiments, the IgA antibody comprises a heavy chain having SEQ ID NO:29. In some embodiments, the IgA antibody comprises a light chain having SEQ ID NO:30. In some embodiments, the IgA antibody comprises a heavy chain having SEQ ID NO:29 and a light chain having SEQ ID NO:30.

In some embodiments, the IgA antibody comprises CDR3: AREWVADDDTFDGFDV (SEQ ID NO:19). In some embodiments, the IgA antibody comprises CDR1: GFIFSAFV (SEQ ID NO:17), CDR2: VWYDGNSK (SEQ ID NO:18), and CDR3: AREWVADDDTFDGFDV (SEQ ID NO:19).

In some embodiments, the IgA antibody comprises CDR3: QQRSNWPPEVT (SEQ ID NO:25). In some embodiments, the IgA antibody comprises CDR1: QSVTNS (SEQ ID NO:23), CDR2: DAS (SEQ ID NO:24) and CDR3: QQRSNWPPEVT (SEQ ID NO:25).

In some embodiments, the IgA antibody comprises CDR3: AREWVADDDTFDGFDV (SEQ ID NO:19) and CDR3: QQRSNWPPEVT (SEQ ID NO:25). In some embodiments, the IgA antibody comprises CDR1: GFIFSAFV (SEQ ID NO:17), CDR2: VWYDGNSK (SEQ ID NO:18), CDR3: AREWVADDDTFDGFDV (SEQ ID NO:19), CDR1: QSVTNS (SEQ ID NO:23), CDR2: DAS (SEQ ID NO:24) and CDR3: QQRSNWPPEVT (SEQ ID NO:25).

In some embodiments, the IgA antibody is a F425-A1g8 antibody.

### Highly Glycosylated Antibodies

In some embodiments, the methods of treating HIV disclosed herein comprise administering to the subject a composition comprising highly glycosylated antibody to treat HIV infection. In some embodiments, the methods of decreasing the chance of HIV infection in a subject comprise administering to the subject a composition comprising highly glycosylated antibody to decrease the chance of HIV infection. In some embodiments, the methods of decreasing the chance of HIV infection in a subject that receives breast milk comprise administering to breast milk a composition comprising highly glycosylated antibody to decrease the chance of HIV infection in a subject that receives the breast milk. In some embodiments, the methods of suppressing mother-to-child transmission of HIV comprise applying a composition comprising highly glycosylated antibody to the nipple of the mother prior to breast feeding to suppress mother-to-child transmission of HIV. In some embodiments, the methods of suppressing mother-to-child transmission of HIV comprise expressing highly glycosylated antibody in one or more cells of the mammary gland of the mother to suppress mother-to-child transmission of HIV. In some embodiments, the methods of suppressing mother-to-child transmission of HIV comprise administering to the mother a composition comprising highly glycosylated antibody to suppress mother-to-child transmission of HIV. In some embodiments, the methods of decreasing the chance of HIV infection in a subject comprise applying to a mucous membrane of the subject a composition comprising highly glycosylated antibody thereby decreasing the chance of HIV infection in the subject.

In one aspect the disclosure provides methods that include the use of compositions comprising highly glycosylated antibodies. Highly glycosylated antibodies, as used herein include antibodies that are at least 3% (w/w) glycosylated, at least 4% (w/w) glycosylated, at least 5% (w/w) glycosylated, at least 6% (w/w) glycosylated, at least 7% (w/w) glycosylated, at least 8% (w/w) glycosylated, at least 9% (w/w) glycosylated, at least 10% (w/w) glycosylated, at least 11% (w/w) glycosylated, at least 12% (w/w) glycosylated, at least 13% (w/w) glycosylated, at least 14% (w/w) glycosylated, at least 15% (w/w) glycosylated, at least 20% or more (w/w) glycosylated.

It should be appreciated that the highly glycosylated antibody can include a variety of glycosylation patterns, and the glycosylation can include fucose, sialic acid, galactose, mannose and other monosaccharide building blocks. In some embodiments, the highly glycosylated antibody includes N-glycosylation. In some embodiments, the highly glycosylated antibody includes O-glycosylation. In some embodiments, the highly glycosylated antibody is an IgA antibody. In some embodiments, the highly glycosylated antibody is a multimeric antibody. In some embodiments, the highly glycosylated antibody is a milk-produced antibody. In some embodiments, the highly glycosylated antibody is produced in the mammary gland of a transgenic non-human mammal. In some embodiments, the highly glycosylated antibody is produced by glycosylating an antibody that is not highly glycosylated.

In some embodiments, the highly glycosylated antibody is a milk-produced antibody. In some embodiments, the highly glycosylated antibody is produced in the mammary gland of a transgenic non-human mammal.

In some embodiments, the highly glycosylated antibody has a glycosylation pattern (i.e., the nature and structure of the glycosylation side chain) that is associated with a milk-produced antibody. In some embodiments, the highly glycosylated antibody has a glycosylation pattern that is associated with antibodies produced in the mammary gland of a transgenic non-human mammal.

In some embodiments, the highly glycosylated antibody is a b12 antibody. In some embodiments, the highly glycosylated antibody is a b12 IgA2 antibody. In some embodiments, the highly glycosylated antibody comprises a heavy chain having SEQ ID NO:29. In some embodiments, the highly glycosylated antibody comprises a light chain having SEQ ID NO:30. In some embodiments, the highly glycosylated antibody comprises a heavy chain having SEQ ID NO:29 and a light chain having SEQ ID NO:30.

In some embodiments, the highly glycosylated antibody comprises CDR3: AREWVADDDTFDGFDV (SEQ ID NO:19). In some embodiments, the highly glycosylated antibody comprises CDR1: GFIFSAFV (SEQ ID NO:17), CDR2: VWYDGNSK (SEQ ID NO:18), and CDR3: AREWVADDDTFDGFDV (SEQ ID NO:19).

In some embodiments, the highly glycosylated antibody comprises CDR3: QQRSNWPPEVT (SEQ ID NO:25). In some embodiments, the highly glycosylated antibody comprises CDR1: QSVTNS (SEQ ID NO:23), CDR2: DAS (SEQ ID NO:24) and CDR3: QQRSNWPPEVT (SEQ ID NO:25).

In some embodiments, the highly glycosylated antibody comprises CDR3: AREWVADDDTFDGFDV (SEQ ID NO:19) and CDR3: QQRSNWPPEVT (SEQ ID NO:25). In some embodiments, the highly glycosylated antibody comprises CDR1: GFIFSAFV (SEQ ID NO:17), CDR2: VWYDGNSK (SEQ ID NO:18), CDR3: AREWVADDDTFDGFDV (SEQ ID NO:19), CDR1: QSVTNS (SEQ ID NO:23), CDR2: DAS (SEQ ID NO:24) and CDR3: QQRSNWPPEVT (SEQ ID NO:25).

In some embodiments, the highly glycosylated antibody is a F425-A1g8 antibody.

### Multimeric Antibodies

In some embodiments, the methods of treating HIV disclosed herein comprise administering to the subject a composition comprising multimeric antibody to treat HIV infection. In some embodiments, the methods of decreasing the chance of HIV infection in a subject comprise administering to the subject a composition comprising multimeric glycosylated antibody to decrease the chance of HIV infection. In some embodiments, the methods of decreasing the chance of HIV infection in a subject that receives breast milk comprise administering to breast milk a composition comprising multimeric antibody to decrease the chance of HIV infection in a subject that receives the breast milk. In some embodiments, the methods of suppressing mother-to-child transmission of HIV comprise applying a composition comprising multimeric antibody to the nipple of the mother prior to breast feeding to suppress mother-to-child transmission of HIV. In some embodiments, the methods of suppressing mother-to-child transmission of HIV comprise expressing multimeric antibody in one or more cells of the mammary gland of the mother to suppress mother-to-child transmission of HIV. In some embodiments, the methods of suppressing mother-to-child transmission of HIV comprise administering to the mother a composition comprising multimeric antibody to suppress mother-to-child transmission of HIV. In some embodiments, the methods of decreasing the chance of HIV infection in a subject comprise applying to a mucous membrane of the subject a composition comprising multimeric antibody thereby decreasing the chance of HIV infection in the subject.

In one aspect the disclosure provides methods that include the use of compositions comprising multimeric antibodies. Multimeric antibodies, as used herein, are antibodies that include multiple immunoglobulins. Multimeric antibodies include IgA (a dimeric antibody) and IgM (a pentameric antibody). However, it should be appreciated that multimeric antibodies are not limited to natural antibodies and include, for instance, multimeric IgG antibodies or other immunoglobulin domains that are coupled together.

In some embodiments, the multimeric antibody is an IgA antibody. In some embodiments, the multimeric antibody is a highly glycosylated antibody. In some embodiments, the multimeric antibody is a milk-produced antibody. In some embodiments, the multimeric antibody is produced in the mammary gland of a transgenic non-human mammal. In some embodiments, the multimeric antibody is produced by glycosylating an antibody that previously was not highly glycosylated.

In some embodiments, the multimeric antibody is a milk-produced antibody. In some embodiments, the multimeric antibody is produced in the mammary gland of a transgenic non-human mammal.

In some embodiments, the multimeric antibody has a glycosylation pattern (i.e., the nature and structure of the glycosylation side chain) that is associated with a milk-produced antibody. In some embodiments, the multimeric antibody has a glycosylation pattern that is associated with antibodies produced in the mammary gland of a transgenic non-human mammal.

It should be appreciated that antibodies can be class-switched. Thus, for instance, IgG antibodies can be class-switched to become multimeric antibodies, including IgA and IgM antibodies. Antibodies directed against a particular antigen are often developed as IgG isotype and subsequently class-switched, such as, for instance, the anti-HIV b12 antibody described herein (See also, Mantis et al. 2007, J. Immunol. 179:3144-3152). Additional anti-HIV antibodies that have been class-switched include 2F5 and 2G12, which have been class-switched from IgG1 to IgM and IgA1 (Wolbank et al. 2003, J. Virol. 77: 4095-4103). A further anti-HIV antibody that has been class-switched is the F425A1g8 antibody (Yu et al. Journal of Immunology 2013, 190: 205-210).

In some embodiments, the multimeric antibody is a b12 antibody. In some embodiments, the multimeric antibody is a b12 IgA2 antibody. In some embodiments, the multimeric antibody comprises a heavy chain having SEQ ID NO:29. In some embodiments, the multimeric antibody comprises a light chain having SEQ ID NO:30. In some embodiments, the multimeric antibody comprises a heavy chain having SEQ ID NO:29 and a light chain having SEQ ID NO:30.

In some embodiments, the multimeric antibody comprises CDR3: AREWVADDDTFDGFDV (SEQ ID NO:19). In some embodiments, the multimeric antibody comprises CDR1: GFIFSAFV (SEQ ID NO:17), CDR2: VWYDGNSK (SEQ ID NO:18), and CDR3: AREWVADDDTFDGFDV (SEQ ID NO:19).

In some embodiments, the multimeric antibody comprises CDR3: QQRSNWPPEVT (SEQ ID NO:25). In some embodiments, the multimeric antibody comprises CDR1: QSVTNS (SEQ ID NO:23), CDR2: DAS (SEQ ID NO:24) and CDR3: QQRSNWPPEVT (SEQ ID NO:25).

In some embodiments, the multimeric antibody comprises CDR3: AREWVADDDTFDGFDV (SEQ ID NO:19) and CDR3: QQRSNWPPEVT (SEQ ID NO:25). In some embodiments, the multimeric antibody comprises CDR1: GFIFSAFV (SEQ ID NO:17), CDR2: VWYDGNSK (SEQ ID NO:18), CDR3: AREWVADDDTFDGFDV (SEQ ID NO:19), CDR1: QSVTNS (SEQ ID NO:23), CDR2: DAS (SEQ ID NO:24) and CDR3: QQRSNWPPEVT (SEQ ID NO:25).

In some embodiments, the multimeric antibody is a F425-A1g8 antibody.

### Milk-Produced Antibodies and Milk

As provided herein, milk-produced antibodies (e.g., IgA antibodies) are more effective in methods of treating HIV infection and decreasing the chance of HIV infection than antibodies that were not milk-produced. In addition, it is shown herein that milk-produced antibodies (e.g., IgA antibodies) in combination with milk show a synergistic effect in the treatment of HIV infection and decreasing the chance of HIV infection.

In some embodiments of the methods and compositions disclosed herein, the antibodies are milk-produced antibodies. In some embodiments, the antibodies are produced in the mammary gland of a transgenic non-human mammal. Milk-produced antibodies, as used herein, refer to antibodies that are produced in mammary epithelial cells or antibodies that are identical to antibodies produced in mammary epithelial cells. Antibodies that are produced in mammary epithelial cells include both antibodies that produced in transgenic animals and antibodies produced in mammary epithelial cells that are cultured (i.e., not in an animal). In general, milk-produced antibodies will have a glycosylation pattern that is different from antibodies that are not produced in milk. Furthermore, the glycosylation pattern may further depend on the animal (e.g., goat or mice) in which the antibody is produced (See e.g., WO2007/048077). It should be appreciated that milk-produced antibodies also include antibodies that are not produced in mammary epithelial cells but that have the same structure, including the same glycosylation pattern as antibodies produced in mammary epithelial cells. Thus including, for instance, antibodies produced in CHO cells that are modified to have the same glycosylation pattern as antibodies produced in mammary epithelial cells.

It is shown herein that milk-produced antibodies (e.g., IgA antibodies) in combination with milk show a synergistic effect in the treatment of HIV infection and decreasing the chance of HIV infection. In some embodiments of the methods and compositions disclosed herein the composition comprises milk. Milk, as used herein, refers to liquid produced in the mammary gland of mammals. Milk is a water-based solution comprising a large amount of casein protein micelles. Milk can be harvested form a variety of mammals and the milk used in the methods and compositions disclosed herein is not limited to a particular animal source. Thus, for instance, the composition can be bovine milk, goat milk and/or mice milk. In some embodiments of the methods and compositions disclosed herein, the composition comprises milk that is produced in the mammary gland of the transgenic animal that produced the antibody (e.g., IgA antibodies). The milk may be added to the composition unpurified, or may be added to the composition after having undergone one or more purification steps (e.g., filtration, microbial inactivation, removal of specific proteins, etc.).

Thus, in one aspect the invention provides compositions comprising milk-produced IgA antibody, multimeric antibody and/or highly glycosylated antibody. In some embodiments, the milk-produced antibody is an IgA antibody. In some embodiments, the milk-produced antibody (e.g., IgA antibody) is produced in the mammary gland of a non-human transgenic mammal. In some embodiments, the composition further comprises milk. In some embodiments, the milk is produced in the mammary gland of a non-human mammal that produces IgA antibody in its mammary gland. In some embodiments, the composition further comprises a pharmaceutically effective antibody.

In some embodiments, the milk-produced antibody is a b12 antibody. In some embodiments, the milk-produced antibody is a b12 IgA2 antibody. In some embodiments, the milk-produced antibody comprises a heavy chain having SEQ ID NO:29. In some embodiments, the milk-produced antibody comprises a light chain having SEQ ID NO:30. In some embodiments, the milk-produced antibody comprises a heavy chain having SEQ ID NO:29 and a light chain having SEQ ID NO:30.

In some embodiments, the milk-produced antibody comprises CDR3: AREWVADDDTFDGFDV (SEQ ID NO:19). In some embodiments, the milk-produced antibody comprises CDR1: GFIFSAFV (SEQ ID NO:17), CDR2: VWYDGNSK (SEQ ID NO:18), and CDR3: AREWVADDDTFDGFDV (SEQ ID NO:19).

In some embodiments, the milk-produced antibody comprises CDR3: QQRSNWPPEVT (SEQ ID NO:25). In some embodiments, the milk-produced antibody comprises CDR1: QSVTNS (SEQ ID NO:23), CDR2: DAS (SEQ ID NO:24) and CDR3: QQRSNWPPEVT (SEQ ID NO:25).

In some embodiments, the milk-produced antibody comprises CDR3: AREWVADDDTFDGFDV (SEQ ID NO:19) and CDR3: QQRSNWPPEVT (SEQ ID NO:25). In some embodiments, the milk-produced antibody comprises CDR1: GFIFSAFV (SEQ ID NO:17), CDR2: VWYDGNSK (SEQ ID NO:18), CDR3: AREWVADDDTFDGFDV (SEQ ID NO:19), CDR1: QSVTNS (SEQ ID NO:23), CDR2: DAS (SEQ ID NO:24) and CDR3: QQRSNWPPEVT (SEQ ID NO:25).

In some embodiments, the milk-produced antibody is a F425-A1g8 antibody.

### Anti-HIV Antibodies

In one aspect the disclosure provides methods of, and compositions for, treating HIV infection and decreasing the chance of HIV infection. In some embodiments, the methods disclosed herein comprise administering to the subject a composition comprising an IgA antibody, a multimeric antibody or a highly glycosylated antibody. In some embodiments, the antibody (e.g., IgA antibody) is a milk-produced antibody. In some embodiments, the milk-produced antibody (e.g., IgA antibody) is produced in the mammary gland of a transgenic mammal. In some embodiments, the composition further comprises milk. In some embodiments, the milk is produced in the mammary gland of a transgenic non-human mammal that produces the antibody (e.g., IgA antibody) in its mammary gland.

In one aspect, the IgA antibodies, multimeric antibodies or highly glycosylated antibodies disclosed herein are anti-HIV antibodies. Anti-HIV antibodies as defined herein are therapeutic antibodies that can bind one or more HIV antigens (e.g., HIV nucleic acids or proteins). Examples of HIV antigens are HIV proteins on the viral particle surface and/or HIV proteins involved with cell entry process, such as gp160, gp120, gp41. Anti-HIV antibodies also include antibodies that bind human targets for HIV proteins (e.g., CCR5, CXCR4).

In some embodiments, the anti-HIV antibody binds gp120. In some embodiments, the anti-HIV antibody binds the CD4 binding site on gp120. In some embodiments, the anti-HIV antibody is a b12 antibody. In some embodiments, the anti-HIV antibody is 2F5. In some embodiments, the anti-HIV antibody is 2G12 (See e.g., Wolbank et al. 2003, J. Virol. 77: 4095-4103). In some embodiments, the anti-HIV antibody is the F425A1g8 antibody (Yu et al. Journal of Immunology 2013, 190: 205-210).

In some embodiments, the anti-HIV antibody is a b12 antibody. In some embodiments, the anti-HIV antibody is a b12 IgA2 antibody. In some embodiments, the anti-HIV antibody comprises a heavy chain having SEQ ID NO:29. In some embodiments, the anti-HIV antibody comprises a light chain having SEQ ID NO:30. In some embodiments, the anti-HIV antibody comprises a heavy chain having SEQ ID NO:29 and a light chain having SEQ ID NO:30.

In some embodiments, the anti-HIV antibody comprises CDR3: AREWVADDDTFDGFDV (SEQ ID NO:19). In some embodiments, the anti-HIV antibody comprises CDR1: GFIFSAFV (SEQ ID NO:17), CDR2: VWYDGNSK (SEQ ID NO:18), and CDR3: AREWVADDDTFDGFDV (SEQ ID NO:19).

In some embodiments, the anti-HIV antibody comprises CDR3: QQRSNWPPEVT (SEQ ID NO:25). In some embodiments, the anti-HIV antibody comprises CDR1: QSVTNS (SEQ ID NO:23), CDR2: DAS (SEQ ID NO:24) and CDR3: QQRSNWPPEVT (SEQ ID NO:25).

In some embodiments, the anti-HIV antibody comprises CDR3: AREWVADDDTFDGFDV (SEQ ID NO:19) and CDR3: QQRSNWPPEVT (SEQ ID NO:25). In some embodiments, the anti-HIV antibody comprises CDR1: GFIFSAFV (SEQ ID NO:17), CDR2: VWYDGNSK (SEQ ID NO:18), CDR3: AREWVADDDTFDGFDV (SEQ ID NO:19), CDR1: QSVTNS (SEQ ID NO:23), CDR2: DAS (SEQ ID NO:24) and CDR3: QQRSNWPPEVT (SEQ ID NO:25).

In some embodiments, the anti-HIV antibody is a F425-A1g8 antibody.

### Treatment of HIV Infection

In one aspect the disclosure provides methods of, and compositions for, treating HIV infection. In some embodiments, the methods of treating HIV disclosed herein comprise administering to the subject a composition comprising an IgA antibody, a multimeric antibody or a highly glycosylated antibody to treat HIV infection. In some embodiments, the antibody (e.g., IgA antibody) is a milk-produced antibody. In some embodiments, the milk-produced antibody (e.g., IgA antibody) is produced in the mammary gland of a transgenic mammal. In some embodiments, the composition further comprises milk. In some embodiments, the milk is produced in the mammary gland of a transgenic non-human mammal that produces the antibody (e.g., IgA antibody) in its mammary gland.

As provided herein, milk-produced antibodies (e.g., IgA antibodies) are more effective in the treatment of HIV than antibodies that were not milk-produced. In addition, it is shown herein milk-produced antibodies (e.g., IgA antibodies) in combination with milk show a synergistic effect in the treatment of HIV infection.

Treating HIV infection, as used herein, includes the change in any physiological parameter that is associated with an improvement in HIV infection. Thus, for instance, treatment of HIV infection includes a decrease in the amount of viral HIV in the blood, a decrease in the amount of infected cells, increase in the amount of white blood cells, etc. Treatment of HIV infection can also be characterized by an increase in immune capacity in a subject and/or a decrease in any condition associated with HIV infection/AIDS (e.g., opportunistic infection, failing immune system, weight loss).

Treatment of HIV infection also includes specific physiological changes such as neutralizing HIV, suppressing ability of HIV to enter cells, suppressing HIV replication and inhibition of the virus, for instance though antibody-dependent cell-mediated viral inhibition (See e.g., Asmal et al., 2011, J. Virology 85: 5465-5475). Such physiological processes can often be evaluated by in vitro and in vivo assays on biological samples (e.g., though a serum sample obtained from an HIV infected person).

In one aspect, the methods and composition used herein are capable of inducing antibody-dependent cell-mediated viral inhibition (ADCVI). Both ADCC (Antibody dependent cell mediated cytotoxicity) and ADCVI are mediated by (HIV-binding) antibodies that interact via their Fc receptors with effector cells, commonly natural killer (NK) cells. The effector cells then destroy infected cells expressing antibody-bound antigen. ADCC is a measure of the ability of effector cells to lyse antibody-bound target cells, while ADCVI describes the ability of virus-specific antibodies and effector cells to inhibit viral replication in a target cell population. Unlike neutralizing antibody responses, which may take months to develop and are initially highly specific to the individual's infecting virus, binding antibodies that mediate ADCC/ADCVI arise early following infection (See e.g., Asmal et al., 2011, J. Virology 85: 5465-5475). In one aspect the methods and composition used herein are capable of inducing antibody-dependent cell-mediated viral inhibition (ADCVI). In one aspect the methods and composition used herein are capable of inducing antibody-dependent cell-mediated cytotoxicity (ADCC). In one aspect the methods and composition used herein are capable of inducing antibody-dependent cell-mediated viral inhibition (ADCVI) and antibody-dependent cell-mediated cytotoxicity (ADCC). While not being limited to a specific mechanism it is though that the antibodies use in the methods and compositions described herein are capable of inducing ADCVI (and ADCC) because they area milk-produced antibodies. In addition, it is shown herein that milk-produced antibodies (e.g., IgA antibodies) in combination with milk show a synergistic effect in the capacity to induce ADCVI.

### Decreasing the Chance of HIV Infection

In one aspect, the disclosure provides methods of, and compositions for, decreasing the chance of HIV infection in a subject, decreasing the chance of HIV infection in a subject that receives breast milk, and suppressing mother-to-child transmission of HIV. In some embodiments, the methods of decreasing the chance of HIV infection in a subject, decreasing the chance of HIV infection in a subject that receives breast milk, and suppressing mother-to-child transmission of HIV disclosed herein comprise administering to the subject a composition comprising an IgA antibody, a multimeric antibody or a highly glycosylated antibody. In some embodiments, the antibody (e.g., IgA antibody) is a milk-produced antibody. In some embodiments, the milk-produced antibody (e.g., IgA antibody) is produced in the mammary gland of a transgenic mammal. In some embodiments, the composition further comprises milk. In some embodiments, the milk is produced in the mammary gland of a transgenic non-human mammal that produces the antibody (e.g., IgA antibody) in its mammary gland.

As provided herein, milk-produced antibodies (e.g., IgA antibodies) are more effective in decreasing the chance of HIV infection in a subject, decreasing the chance of HIV infection in a subject that receives breast milk, and suppressing mother-to-child transmission of HIV than antibodies that were not milk-produced. In addition, it is shown herein milk-produced antibodies (e.g., IgA antibodies) in combination with milk show a synergistic effect in decreasing the chance of HIV infection in a subject, decreasing the chance of HIV infection in a subject that receives breast milk, and suppressing mother-to-child transmission of HIV.

Decreasing the chance of HIV infection in a subject, as used herein, means a decrease in the chance a subject would become infected with HIV by using one or more of methods disclosed herein in comparison to a control (e.g., not administering the compositions disclosed herein). For instance, a subject may have specific chance (e.g., 25%, 20%, 15%, etc.) of becoming infected with HIV upon receiving a blood transfusion with blood that includes HIV, upon being exposed to seminal fluid that includes HIV, or upon receiving breast milk that includes HIV. In one aspect, the chance of becoming exposed is decreased by applying the methods disclosed herein (e.g., it may decrease from 25% to 10%, from 10% to 5%, or from 10% to less than 1%). There are a variety of ways in which the chance of infection (and a decrease in the chance of infection) can be monitored and evaluated. For instance, experiments in group of primates can provide the chance of a subject becoming infected to HIV after being exposed to HIV. In addition, the methods disclosed herein can be evaluated in in vitro experiments. For instance, experiments in cells can reveal if a particular method decreases the number of cells that are being infected by HIV (and thus allow for the evaluation if a specific method is effective in decreasing the chance of HIV infection in a subject).

At the end of 2008, 2.1 million children under the age of 15 were HIV infected with the majority of these individuals contracting the virus from their infected mother. Transmission of HIV by breastfeeding accounts for approximately 40% of Mother-To-Child Transmission (MTCT). Infection by HIV can occur through breaks in the integrity of the epithelium, which may occur as a result of inflammation when infants are not exclusively breastfed or are exposed to pathogens. The mucosal barrier, as well as anti-viral properties of some components of breast milk, prevents the majority of infants from becoming infected despite repeated daily exposures to HIV. However, once the mucosal barrier has been crossed, HIV targets resting T cells and disseminates to the draining lymph nodes and the lymphoid.

Maternal HIV specific antibodies in the form of secretory IgA, secretory IgM and IgG are found in breast milk. Given the multitude of components of breast milk which may vary with time, as well as differences in assay methodology, it remains unclear how effective the HIV specific antibody, induced during natural infection, is at preventing transmission^{3,4}. However, breast milk antibodies and the humoral immune response play a significant role in the control of a number of human viral diseases. Since maternal antibodies generally do not enter the circulation of infants through the gastrointestinal tract, they may function to prevent infection by neutralizing viral inoculum or preventing transmission across the epithelial cells either by immune exclusion or intracellular neutralization. Given the more active role of IgA antibodies in mucosal secretions, as compared to IgG antibodies, anti-HIV IgA antibodies at the mucosal surface can decrease the chance of HIV infection.

In one aspect, the disclosure provides methods of, and compositions for, decreasing the chance of HIV infection in a subject that receives breast milk comprising administering to breast milk a composition comprising an IgA antibody, a multimeric antibody or a highly glycosylated antibody. While not being limited to a specific embodiment, it is envisioned that the breast milk would be pumped by the mother (e.g., or wet nurse). The milk could be pumped for instance with a hand-operated mechanical device or an electric pump. The composition comprising the antibody could be administered immediately after the pumping of the breast milk and the breast milk including the composition administered (i.e., fed) to a subject (e.g., a child). However, the breast milk could be harvested and stored e.g., in a fridge or freezer, and the composition comprising the antibody could be administered prior to feeding. Also, the breast milk could be harvested and the composition comprising the antibody could be added and the combination could be stored e.g., in a fridge or freezer, for a specific time prior to feeding.

In one aspect, the disclosure provides methods of, and compositions for, suppressing mother-to-child transmission of HIV, comprising administering to the nipple of the mother prior to breast feeding a composition comprising an IgA antibody, a multimeric antibody or a highly glycosylated antibody. In some embodiments, the composition comprising the antibody is applied to the nipple of the mother (or wet nurse) prior to breast feeding the child. In some embodiments, the composition comprising the antibody is applied to the nipple of the mother (or wet nurse) when the breast milk is pumped (i.e., harvested). Topical ointment and crèmes for applying the compositions disclosed herein the nipple are known in the art and are also described in the administration section below.

In one aspect, the disclosure provides methods of, and compositions for, suppressing mother-to-child transmission of HIV, comprising expressing an IgA antibody, a multimeric antibody or a highly glycosylated antibody in one or more cells of the mammary gland of the mother. While not being limited to a specific technique the antibody could be expressed by introducing nucleic acid encoding the antibody in the mammary gland through gene therapy and/or through injection and/or topical application.

In one aspect, the disclosure provides methods of, and compositions for, suppressing mother-to-child transmission of HIV, comprising administering to the mother a composition comprising an IgA antibody, a multimeric antibody or a highly glycosylated antibody.

It should be appreciated that mother, as used herein includes both the biological mother and a wet nurse (i.e., a person providing breast milk). However, in some embodiments, the mother is the biological mother (i.e., not a wet nurse).

In one aspect the disclosure provides methods of, and compositions for, decreasing the chance of HIV infection in a subject comprising applying to the mucous membrane a composition comprising an IgA antibody, a multimeric antibody or a highly glycosylated antibody. Mucous membranes predominantly found in organs controlling absorption and secretion. Example of mucous membranes include the buccal mucosa, the esophageal mucosa, the gastric mucosa, the intestinal mucosa, the nasal mucosa, the olfactory mucosa, the oral mucosa, the bronchial mucosa, the uterine mucosa, the endometrium (mucosa of the uterus) and the penile mucosa. Topical ointments, crèmes and pharmaceutical compositions for applying the compositions disclosed herein to mucous membranes are known in the art and are also described in the administration section below. In some embodiments, the composition is applied in a vaginal crème.

### Subject

A “subject”, as used herein, is a mammal, such as a human, non-human primate, cow, dog, cat, horse, etc. In some embodiments, the subject is a human. In some embodiments, the subject is a child. In some embodiments, the subject receives breast milk. In some embodiments, the subject is nursing. In some embodiments, the subject is a mother. In some embodiments, the mother is not a wet nurse.

### Therapeutically Effective Amount

In one aspect, the disclosure provides methods of treating HIV and decreasing the chance of HIV infection in a subject, and compositions used in these methods. It is envisioned that in the methods disclosed herein the compositions are used in therapeutically effective amount to achieve the desired results (e.g., treating HIV infection and/or decreasing the chance of HIV infection in a subject).

The terms “therapeutically effective amount” and “effective amount”, which are used interchangeably, refer to the amount necessary or sufficient to realize a desired therapeutic effect, e.g., treating HIV infection and/or decreasing the chance of HIV infection in a subject. Combined with the teachings provided herein, by choosing among the various active compounds and weighing factors such as potency, relative bioavailability, subject body weight, severity of adverse side-effects and preferred mode of administration, an effective prophylactic or therapeutic treatment regimen can be selected which does not cause substantial toxicity and yet is effective to treat the particular subject.

The effective amount for any particular application can vary depending on such factors as the disease or condition being treated, the particular therapeutic agent(s) to be administered, the size of the subject, or the severity of the disease or disorder. One of ordinary skill in the art can empirically determine the effective amount of the compositions (e.g., compositions comprising milk-produced IgA antibody) without necessitating undue experimentation. It is preferred generally that a maximum dose be used, that is, the highest safe dose according to some medical judgment. Multiple doses per day, week or month may be contemplated to achieve appropriate systemic levels of the administered compositions (and its components). Appropriate system levels can be determined by, for example, measurement of the patient's peak or sustained plasma level of the components of the compositions, such as milk-produced IgA antibody.

Doses of the compositions to be administered in the methods disclosed herein can vary depending on the desired therapeutic goal. In some embodiments, the composition is administered at a dose sufficient to treat HIV infection. In some embodiments, the composition is administered at a dose sufficient to decrease the chance of HIV infection in a subject.

In some embodiments, the therapeutically effective amount is administered in one dose. In some embodiments, the therapeutically effective amount is administered in multiple doses. Dosage may be adjusted appropriately to achieve desired levels of the composition, local or systemic, depending upon the mode of administration. In the event that the response in a subject is insufficient at such doses, even higher doses (or effective higher doses by a different, more localized delivery route) may be employed to the extent that subject tolerance permits. Multiple doses per day may be contemplated to achieve appropriate systemic levels of compounds.

### Additional Anti-HIV Therapies

In some embodiments, the methods of treatment provides herein are combined with anti-viral therapies (e.g., anti-HIV therapies). Anti-viral therapies and compounds used therein include compounds that suppress or inhibit viral infection, viral proliferation and/or the development of disease associated with viral infection. Anti-viral drugs can be classified as targeting one of the life cycle stages of the virus. One category of anti-viral drugs is based on interfering with viral entry. A virus binds to a specific receptor to infiltrate a target cell. Viral entry can be suppressed by blocking of the viral entry way. Anti-viral drugs that have this mode of action are anti-receptor antibodies, natural ligands of the receptor and small molecules that can bind to the receptor. A second category of antiviral drugs are compounds that suppress viral synthesis. Antiviral drugs that have this mode of action are nucleoside analogues that are similar to the DNA and RNA building blocks but deactivate the protein machinery (e.g., reverse transcriptase or DNA polymerase) used to replicate the virus. Other drugs are targeted at blocking the transcription factors of viral DNA, ribozymes, which can interfere with the production of viral DNA. Other drugs target viral RNA for destruction, including siRNAs and antisense nucleic acids against viral nucleic acid sequences. Yet another class of antiviral drugs includes drugs that can interfere with the function of virus specific proteins. This class includes the HIV protease inhibitors. Antiviral drugs also include drugs directed at the release stage if the virus. This category of drugs includes compounds that interfere with the proteins necessary to build the viral particles. Another class of antiviral drugs includes drugs that stimulate the immune system in targeting viral infection. Drugs that fall in this class are interferons, which inhibit viral synthesis in infected cells and antibodies that can target an infected cell for destruction by the immune system. Other anti-viral agents are described in ford instance U.S. Pat. Nos. 6,130,326, and 6,440,985, and published US patent application 20020095033.

In some embodiments, the methods disclosed herein include anti-retroviral therapy directed against HIV, including HAART (Highly Active Antiretroviral Therapy). Antiretroviral (ARV) drugs include entry inhibitors (or fusion inhibitors), CCR5 receptor antagonists, nucleoside reverse transcriptase inhibitors (NRTI), nucleotide reverse transciptase inhibitors (NtRTI), non-nucleoside reverse transcriptase inhibitors (NNRTI), protease inhibitors (PIs) and integrase inhibitors. Commercially available anti-HIV drugs and HIV drug combinations include zidovudine, lamivudine, abacavir, combivir, trizivir, enfuvirtide, kaetra, truvada, opinavir and ritonavir.

Constructs for the Generation of Transgenic Animals Expressing Antibodies into Milk

A DNA sequence which is suitable for directing production of an antibody to the milk of transgenic animals can carry a 5′-promoter region derived from a naturally-derived milk protein. This promoter is consequently under the control of hormonal and tissue-specific factors and is most active in lactating mammary tissue. In some embodiments, the promoter is a caprine beta casein promoter. The promoter can be operably linked to a DNA sequence directing the production of a protein leader sequence, which directs the secretion of the transgenic protein across the mammary epithelium into the milk. In some embodiments, a 3′-sequence, which can be derived from a naturally secreted milk protein, can be added to improve stability of mRNA.

As used herein, a “leader sequence” or “signal sequence” is a nucleic acid sequence that encodes a protein secretory signal, and, when operably linked to a downstream nucleic acid molecule encoding a transgenic protein directs secretion. The leader sequence may be the native human leader sequence, an artificially-derived leader, or may obtained from the same gene as the promoter used to direct transcription of the transgene coding sequence, or from another protein that is normally secreted from a cell, such as a mammalian mammary epithelial cell.

In some embodiments, the promoters are milk-specific promoters. As used herein, a “milk-specific promoter” is a promoter that naturally directs expression of a gene in a cell that secretes a protein into milk (e.g., a mammary epithelial cell) and includes, for example, the casein promoters, e.g., α-casein promoter (e.g., alpha S-1 casein promoter and alpha S2-casein promoter), β-casein promoter (e.g., the goat beta casein gene promoter (DiTullio, BIOTECHNOLOGY 10:74-77, 1992), γ-casein promoter, κ-casein promoter, whey acidic protein (WAP) promoter (Gordon et al., BIOTECHNOLOGY 5: 1183-1187, 1987), β-lactoglobulin promoter (Clark et al., BIOTECHNOLOGY 7: 487-492, 1989) and α-lactalbumin promoter (Soulier et al., FEBS LETTS. 297:13, 1992). Also included in this definition are promoters that are specifically activated in mammary tissue, such as, for example, the long terminal repeat (LTR) promoter of the mouse mammary tumor virus (MMTV).

### Transgenic Animals

In one aspect, the disclosure provides mammary gland epithelial cells that express the antibodies (e.g., IgA antibodies) used in the methods and compositions of the disclosure. In some embodiments, the disclosure provides a transgenic non-human mammal comprising the mammary gland epithelial cells mammary gland epithelial cells that express these antibodies

In one aspect, the disclosure provides a method for the production of a transgenic antibody, and variants and fragments thereof, the process comprising expressing in the milk of a transgenic non-human mammal a transgenic antibody encoded by a nucleic acid construct. In some embodiments, the method for producing the antibodies of the invention comprises:

(a) transfecting non-human mammalian cells with a transgene DNA construct encoding a desired transgenic antibody; (b) selecting cells in which said transgene DNA construct has been inserted into the genome of the cells; and (c) performing a first nuclear transfer procedure to generate a non-human transgenic mammal heterozygous for the desired transgenic antibody and that can express it in its milk.

In one aspect, the disclosure provides a method of (a) providing a non-human transgenic mammal engineered to express an antibody, (b) expressing the antibody in the milk of the non-human transgenic mammal; and (c) isolating the antibodies expressed in the milk. Such methods can further comprise steps for inducing lactation.

Transgenic animals, capable of recombinant antibody expression, can also be generated according to methods known in the art (See e.g., U.S. Pat. Nos. 5,349,992 and 5,945,577, WO2004//050847 and Sola et al., 1998, J. of Virology 72: 3762-3772). Animals suitable for transgenic expression, include, but are not limited to goat, sheep, bison, camel, cow, pig, rabbit, buffalo, horse, rat, mouse or llama. Suitable animals also include bovine, caprine, ovine and porcine, which relate to various species of cows, goats, sheep and pigs (or swine), respectively. Suitable animals also include ungulates. As used herein, “ungulate” is of or relating to a hoofed typically herbivorous quadruped mammal, including, without limitation, sheep, swine, goats, cattle and horses. In one embodiment, the animals are generated by co-transfecting primary cells with separate constructs containing the heavy and light chains. These cells are then used for nuclear transfer. Alternatively, if micro-injection is used to generate the transgenic animals, the constructs may be-injected.

Cloning will result in a multiplicity of transgenic animals—each capable of producing an antibody or other gene construct of interest. The production methods include the use of the cloned animals and the offspring of those animals. In some embodiments, the cloned animals are caprines, bovines or mice. Cloning also encompasses the nuclear transfer of fetuses, nuclear transfer, tissue and organ transplantation and the creation of chimeric offspring.

One step of the cloning process comprises transferring the genome of a cell that contains the transgene encoding the antibody into an enucleated oocyte. As used herein, “transgene” refers to any piece of a nucleic acid molecule that is inserted by artifice into a cell, or an ancestor thereof, and becomes part of the genome of an animal which develops from that cell. Such a transgene may include a gene which is partly or entirely exogenous (i.e., foreign) to the transgenic animal, or may represent a gene having identity to an endogenous gene of the animal.

Suitable mammalian sources for oocytes include goats, sheep, cows, pigs, rabbits, guinea pigs, mice, hamsters, rats, non-human primates, etc. Preferably, oocytes are obtained from ungulates, and most preferably goats or cattle. Methods for isolation of oocytes are well known in the art. Essentially, the process comprises isolating oocytes from the ovaries or reproductive tract of a mammal, e.g., a goat. A readily available source of ungulate oocytes is from hormonally-induced female animals. For the successful use of techniques such as genetic engineering, nuclear transfer and cloning, oocytes may preferably be matured in vivo before these cells may be used as recipient cells for nuclear transfer, and before they were fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone.

One of the tools used to predict the quantity and quality of the recombinant protein expressed in the mammary gland is through the induction of lactation (Ebert K M, 1994). Induced lactation allows for the expression and analysis of protein from the early stage of transgenic production rather than from the first natural lactation resulting from pregnancy, which is at least a year later. Induction of lactation can be done either hormonally or manually.

In some embodiments, the compositions of antibodies provided herein further comprise milk. In some embodiments, the methods provides herein includes a step of isolating the population of antibodies from the milk of a transgenic animal. Methods for isolating antibodies from the milk of transgenic animal are known in the art and are described for instance in Pollock et al., Journal of Immunological Methods, Volume 231, Issues 1-2, 10 Dec. 1999, Pages 147-157.

### Administration

In one aspect, the disclosure provides methods of treating HIV and decreasing the chance of HIV infection in a subject, and compositions used in these methods. The compositions (e.g., comprising, milk-produced IgA antibodies, multimeric antibodies or highly glycosylated antibodies) are typically administered to subjects as pharmaceutical compositions, which may routinely contain pharmaceutically acceptable concentrations of salt, buffering agents, preservatives, compatible carriers, adjuvants, and optionally other therapeutic ingredients. The nature of the pharmaceutical carrier and other components of the pharmaceutical composition will depend on the mode of administration.

The pharmaceutical compositions of the disclosure may be administered by any means and route known to the skilled artisan in carrying out the treatment methods described herein. Preferred routes of administration include but are not limited to oral, intravenous, subcutaneous, parenteral, intratumoral, intramuscular, intranasal, intracranial, sublingual, intratracheal, inhalation, ocular, vaginal, and rectal. In some embodiments, the compositions are administered topically, e.g., by applying to the nipple.

The compositions, when it is desirable to deliver systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

For oral administration, the compositions can be formulated readily by combining the compounds with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally, the oral formulations may also be formulated in saline or buffers, e.g., EDTA for neutralizing internal acid conditions, or may be administered without any carriers.

For oral delivery, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Examples of the more common inert ingredients that are used as enteric coatings are cellulose acetate trimellitate (CAT), hydroxypropylmethyl-cellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films. A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic powder; for liquid forms, a soft gelatin shell may be used. The shell material of cachets could be thick starch or other edible paper. For pills, lozenges, molded tablets or tablet triturates, moist massing techniques can be used.

Compositions can be included in the formulation as fine multi-particulates in the form of granules or pellets. The formulation of the material for capsule administration could also be as a powder, lightly compressed plugs or even as tablets. The pharmaceutical composition could be prepared by compression. One may dilute or increase the volume of the pharmaceutical composition with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the pharmaceutical composition, such as in a solid dosage form. Materials used as disintegrants include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may also be used. Binders may be used to hold the therapeutic agent together to form a hard tablet and include materials from natural products such as acacia, tragacanth, starch and gelatin. An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Glidants that might improve the flow properties of the drug during formulation and to aid rearrangement during compression might be added. The glidants may include starch, talc, pyrogenic silica and hydrated silicoaluminate.

For administration by inhalation, the compositions may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas.

Also contemplated herein is pulmonary delivery of the compositions. The compositions may be delivered to the lungs of a mammal for local or systemic delivery. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Nasal delivery of the (pharmaceutical) compositions is also contemplated. Nasal delivery allows the passage of a pharmaceutical composition to the blood stream directly after administering the composition to the nose, without the necessity for deposition of the product in the lung.

In some embodiments, the compositions are administered locally. Local administration methods are known in the art and will depend on the target area or target organ. Local administration routes include the use of standard topical administration methods such as epicutaneous (application onto the skin), by inhalational, rectal (e.g., by enema or suppository), by eye drops (onto the conjunctiva), ear drops, intranasal route, and vaginal. In some embodiments, to compositions are formulated for application to the nipple (e.g., prior to breast feeding).

The compositions may also be formulated in rectal or vaginal compositions such as suppositories or retention enemas and vaginal cremes, e.g., containing conventional suppository bases such as cocoa butter or other glycerides. In addition to the formulations described previously, the compounds may also be formulated as a depot preparation. Such long acting formulations may be formulated with suitable polymeric or hydrophobic materials (for example as emulsion in an acceptable oil) or ion exchange resins, or as sparingly soluble analogs, for example, as a sparingly soluble salt. In some embodiments, the composition is administered as a vaginal crème. For instance, the composition can be administered (e.g., applied) when there is a chance of transmission of HIV, e.g. prior to sexual intercourse.

The pharmaceutical compositions also may comprise suitable solid or gel phase carriers or excipients. Examples of such carriers or excipients include but are not limited to calcium carbonate, calcium phosphate, various sugars, starches, cellulose analogs, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or one or more auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, 1990, Science 249, 1527-1533, which is incorporated herein by reference.

The agents and compositions described herein may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

The pharmaceutical compositions of the disclosure contain the compositions (e.g., including a milk-produced IgA antibody) and a pharmaceutically-acceptable carrier. The term pharmaceutically acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compositions of the present disclosure, and with each other, in a manner such that there is no interaction which would substantially impair the desired pharmaceutical efficiency.

Both non-biodegradable and biodegradable polymeric materials can be used in the manufacture of particles for delivering the compositions of the disclosure. Such polymers may be natural or synthetic polymers. The polymer is selected based on the period of time over which release is desired. Bioadhesive polymers of particular interest include bioerodible hydrogels described by Sawhney et al., 1993, Macromolecules 26, 581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The compositions may be contained in controlled release systems. The term “controlled release” is intended to refer to any agents and compositions described herein-containing formulation in which the manner and profile of agents and compositions described herein release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a compound over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the compound therefrom. “Delayed release” may or may not involve gradual release of a compound over an extended period of time, and thus may or may not be “sustained release.” Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

### Kits

In one aspect, the disclosure provides kits comprising the compositions disclose herein (e.g., comprising milk-produced IgA antibodies). In some embodiments, the composition is in sterile container(s). In some embodiments, the kit comprises a pharmaceutical carrier and instructions for administration of the kit components. In some embodiments, the kit includes a pharmaceutical preparation vial, a pharmaceutical preparation diluent vial, and the composition. The diluent vial contains a diluent such as physiological saline for diluting what could be a concentrated solution or lyophilized powder of a composition of the disclosure. In some embodiments, the instructions include instructions for mixing a particular amount of the diluent with a particular amount of a concentrated pharmaceutical composition, whereby a final formulation for injection or infusion is prepared. In some embodiments, the instructions include instructions for use in a syringe or other administration device. In some embodiments, the instructions include instructions for treating a patient with an effective amount of a composition of the disclosure. It also will be understood that the containers containing the preparations, whether the container is a bottle, a vial with a septum, an ampoule with a septum, an infusion bag, and the like, may contain indicia such as conventional markings which change color when the preparation has been autoclaved or otherwise sterilized.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1

### Materials and Methods

### Monoclonal Antibodies, Virus and Cell Lines:

The neutralizing antibody IgG1b12 (b12), directed to the CD4 binding site of gp120, was originally isolated in the laboratory of Dr. Dennis Burton of The Scripps Research Institute from an antibody phage display library prepared from an asymptomatic HIV-1-seropositive individual⁶. CHO-K1 cells were from American Type Culture Collection. The following reagents were obtained through the AIDS Research and Reference Reagent Program, Division of AIDS, NIAID, NIH: SF162 (R5) from Dr. Jay Levy; 92HT593 (R5X4) from Dr. Neal Halsey; 89.6 (R5X4) from Dr. Ronald Collman; BaL (R5) from Dr. Suzanne Gartner, Dr. Mikulas Popovic and Dr. Robert Gallo; 93MW960 (clade C, R5) from Dr. Robert Bollinger and the UNAIDS Network for HIV; JR-FL (R5) from Dr. Irvin Chen; TZM-b1 cells from Dr. John C. Kappes, Dr. Xiaoyun Wu and Transzyme, Inc. Isolate 67970 (CXCR4) was from Dr. David Montefiori.

### Construction and Production of Anti-HIV b12 IgG1 and IgA Variants

b12 VH and VL were PCR amplified respectively from b12 clones from Dr. Burton (The Scripps Research Institute, La Jolla, Calif.) using the specific primers to introduce restriction enzymes (5′Nhe I and 3′Hind III for VH; 5′Nhe I and 3′Not I for VL). The b12 VH fragment was cloned into the immunoglobulin expression vectors pHC-huCg1 and pHC-huCg1. The b12 VL was cloned into vector pLC-huCk. All of these immunoglobulin expression vectors were obtained from Dr. Gary McLean, containing the light chain, γ1 or α1 constant regions, respectively. For constructing b12 IgA2, the α2 constant region from an IgA2 vector (6425pAH-ETEC6-IgA2m2, allotype m2) provided by Dr. Sherrie Morrison was amplified. b12 VH was then connected to the 5 end of a2 constant region by over-lap PCR. Unique restriction sites, 5′Nhe and 3′Xho I, were added to the fragment and replaced the b12A1 fragment in pHC-huCα1 vector using these two restriction enzymes.

To facilitate clonal expression, the complete immunoglobulin cassettes were cloned into IRES-based bicistronic expression vectors (Clontech). b12VL+Ck fragment from pLC-huCk was cloned into pIRESneos3 vector using restriction sites 5′Nhe I and 3′Xba I. Heavy chain fragments of b12VH-Cγ1, b12VH-Cα1 and b12VH-Cα2 were respectively cloned into pIRESpuro3 vector using restriction sites 5′Nhe I and 3′Xho I. The plasmids carrying the b12 light chain and heavy chain were purified with the Maxiprep Kit (Qiagen). Purified plasmid encoding the b12 light chain was then transfected into CHO-K1 cells by lipofection (Invitrogen Life Technologies). After about two weeks selected by RPMI 1640 containing 800 μg/ml G418, the expressing clones were isolated using human κ chain capture ELISA. Cell lines expressing the b12 light chain were propagated and subsequently transfected with the IRESpuro3 plasmids encoding b12 heavy chains of the IgG1, IgA1 and IgA2 class individually. After around 2 week selected with medium containing 10 μg/ml puromycin and 800 μg/ml G418, the expressing cell clones that generated mature subclass b12 were isolated by subclass specific ELISA.

Construction and Production of b12dIgA1

J chain was isolated from human heteromyeloma cells HMMA2.5⁷ by Reverse Transcription PCR (RT-PCR) using primers derived from a human J chain sequence deposited in GenBank (accession number AAH38982). Forward primer was CTAGCTAGCATGAAGAACCATTTGC (SEQ ID NO:1) and reverse primer was TGCGATATCTTAGTCAGGATAGCAGG (SEQ ID NO:2). The restriction sites 5′Nhe I and 3′EcoR V were added in primers. The J chain fragment obtained from PCR was cloned into pcDNA3.1 vector using Nhe I and EcoR V restriction sites. The purified J chain plasmid DNA was transfected into the cell line of b12A1/CHO by lipofection. The selected medium included 800 μg/ml G418, 10 μg/ml puromycin and 1 mg/ml zeocin. The positive clones were screened by J chain ELISA. Briefly, ELISA plate was coated using Goat anti-human κ chain and blocked using 0.1% BSA/PBS buffer. Bound antibody was detected using mouse anti-human J chain antibody followed by HRP conjugated goat anti-mouse IgG antibody.

### Construction of b12 IgA2 Milk Specific Vectors and Expression in Mice:

The expression plasmids containing the b12 heavy and light chains were used as a source of the DNA to construct milk specific expression vectors. Restriction sites were introduced immediately upstream and downstream of the coding sequences, so that they could be cloned into the GTC goat β-casein expression vector. This vector carries the promoter and the downstream untranslated region of goat β-casein (pBC1, Invitrogen), and directs expression of linked genes to the mammary gland with subsequent secretion into the milk.

Two different versions of this vector were used. The gbc450 has the prokaryotic sequences flanked by Sal I sites, while gbc451 has them flanked by Not I sites. The prokaryotic sequences are removed prior to generation of transgenic animals. The upstream Nhe I and downstream Not I sites flanking the IgA2 heavy chain were converted to Sal I sites. The heavy chain could then be retrieved as a Sal I fragment, and ligated into the Xho I cloning site in the milk specific vector gbc451 to yield BC2470HC. The light chain was also subcloned by first changing the upstream Nhe I site to an Xho I site. The Xho I fragment containing the light chain was then ligated into the Xho I cloning site of the modified casein vector gbc450 to yield BC2526LC. These plasmids were used as sources of DNA to generate transgenic mice. Following removal of prokaryotic sequences, the DNA fragments were co-injected into pre-implantation mouse embryos and implanted into foster mothers. The resulting progeny were screened for the presence of the transgenes in their DNA. Female mice that carried the integrated beta casein linked to heavy and light chains of the antibody were grown to maturity and bred. Following parturition, the animals' milk was collected and analyzed for the presence of the b12 antibody. All animal studies were approved by the GTC IACUC.

### Neutralization Assay

The neutralization activity of b12 variants was determined in vitro using a TZM-b1 assay with a panel of five isolates, including an R5/clade C (93MW960) as well as SF162, JR-FL, 89.6 and 67970. Primary isolate virus was grown in PHA-stimulated peripheral blood mononuclear cells (PBMC) as previously described, and detected titer on TZM-b1 cells⁸ to determine TCID50. Serial two-fold dilutions of b12 variants were incubated with virus stock diluted to 100TCID₅₀ for 1 hour, 37° C. prior to the addition of TZM-b1 cells (1×10⁴ c/well). Plates were incubated for 48 hours, 37° C., 5% CO2 prior to measurement of β-galactosidase activity by using β-galactosidase reagent from Promega as an indicator of HIV replication. Percent neutralization was determined based on control wells of virus, media, IC₅₀ and IC₉₀ values calculated by regression analysis.

### Antibody Dependent Cell-Mediated Viral Inhibition (ADCVI)

ADCVI activity was measured using HIV grown in PHA stimulated PBMC as previously described. Neutrophils were obtained from peripheral blood of seronegative donors by Ficoll-Hypaque gradient centrifugation. Antibodies were titered in 96 well, round bottom plates in 50 μl media containing 20% heat-inactivated FBS. Target cells were PBMC productively infected with HIV-1 four days prior to use as previously described⁹ and 1×10⁵ infected cells in 50 μl were added per well. Within 10 minutes of combining of antibody and infected cells, neutrophils were added to the wells at 1×10⁶ effector cells/well in 100 resulting in an effector to target, ratio of 10:1 (E:T). After 4 hours, in order to measure the surviving infectious virus, PHA stimulated PBMC were added as indicator cells (1×10⁵/well). These indicator PBMC were incubated for seven days in the presence of IL-2, at which time ELISA was used to quantitate p24⁷. IC₅₀ values were determined by linear regression analysis and significance was ascertained by Student's t test. Control wells included no antibody, no effectors and no targets were used to determine background release of virus, maximum production of virus, or whether PMN alone were infected, respectively. Experiments were repeated three times.

### Results:

### Construction and Expression of b12 Isotype Variants in CHO Cells

The monoclonal antibody b12 was isotype switched and expressed as an IgG1 IgA1, IgA2, and dimeric IgA1 (dIgA). For constructing isotype variants, immunoglobulin expression vectors were obtained from Dr. Gary McLean for light chain and γ1 or α1 constant regions. PCR products encompassing the variable regions of the light and heavy chains of the b12 antibody were cloned in frame into immunoglobulin expression vectors. A vector expressing α2 was generated by replacing the α1 constant region in the McLean vector with the IgA2 constant region which was isolated from an IgA2 vector (6425pAH-ETEC6-IgA2m2, allotype m2) provided by Dr. Sherrie Morrison. Since restriction site Hind III was not a unique site in A2 constant region, the b12 VH was connected to the A2 constant region using the overlap PCR technique. The whole b12A2 fragment was then cloned into the pHC-huCα1 vector to replace the b12A1 fragment with Nhe I and Xho I restriction sites. Following expression vector construction, the sequence all of plasmid DNA was verified by dideoxy sequencing.

To obtain the b12 isotype variant clonal expression, the complete immunoglobulin cassettes were cloned into IRES-based bicistronic expression vectors (Clontech). The vectors were used because both the gene of interest and the antibiotic resistance gene are encoded by the same mRNA, which not only facilitates clonal selection, but also maintains constant protein expression over time since the selective pressure is exerted on the entire expression cassette¹⁰. The b12 isotype variant clones were identified producing immunoglobulin at concentrations ranging from 1-30 μg/mL by κ chain capture ELISA. All antibodies were purified using protein L columns and quantitated using known concentrations of κ chain.

Clones expressing dIgA1 were derived by sequentially transfecting the J chain into a CHO cell line that was co-expressing b12 light chain and b12 α1 heavy chain. J chain is a 15 kDa polypeptide covalently linked to the C-terminus of two IgA monomers. It is responsible for the intracellular assembly of IgA by modulating their structures and thereby effector functions. J chain is absolutely required for IgA polymerization^{11,12}.

Prior to functional assays, comparable immunoreactivity with gp120 was confirmed by ELISA. All clones displayed significant and comparable reactivity to HIVgp120, as assessed by ELISA (data not shown). Switch variants of b12 (IgG1, IgA1, IgA2 and dIgA1) were compared for neutralization of HIV using TZM-b1 indicator cells. As shown in Table I, the IgA2 subclass and dimeric IgA1 required slightly more antibody for effective neutralization; however, this was not consistent across all isolates and all constructs retained neutralization activity.

TABLE I
Neutralization of HIV-1 by b12 Isotype Variants^{a}

| | IC₉₀ | IC₉₀ | IC₉₀ | IC₉₀ | IC₅₀ |
|---|---|---|---|---|---|
| | SF162 | JR-FL | 89.6 | 93MW960 | 67970 |
| IgG1 | 4.2 (1.4)^{b} | 6.6 (1.0) | 15.1 (5.1) | 12.9 (3.8) | 5.8 (1.5) |
| IgA1 | 4.5 (1.5) | 7.5 (1.4) | 25.4 (10.1) | 33.4 (8.1) | 10.7 (3.4) |
| IgA2 | 6.7 (0.3) | 10.3 (1.7) | 37.7 (5.9) | 18.2 (1.5) | 13.7 (1.9) |
| dIgA1 | 4.4 (1.7) | 12.6 (1.5) | 33.4 (8.1) | 15.1 (2.6) | 11.3 (2.5) |

^{a}Neutralization of HIV was measured in TZM-bl cells and IC90 or IC50 determined by linear regression
^{b}Mean IC90 or IC50 with standard deviation in parenthesis

### Production of b12 IgA2 in the Milk of Transgenic Mice

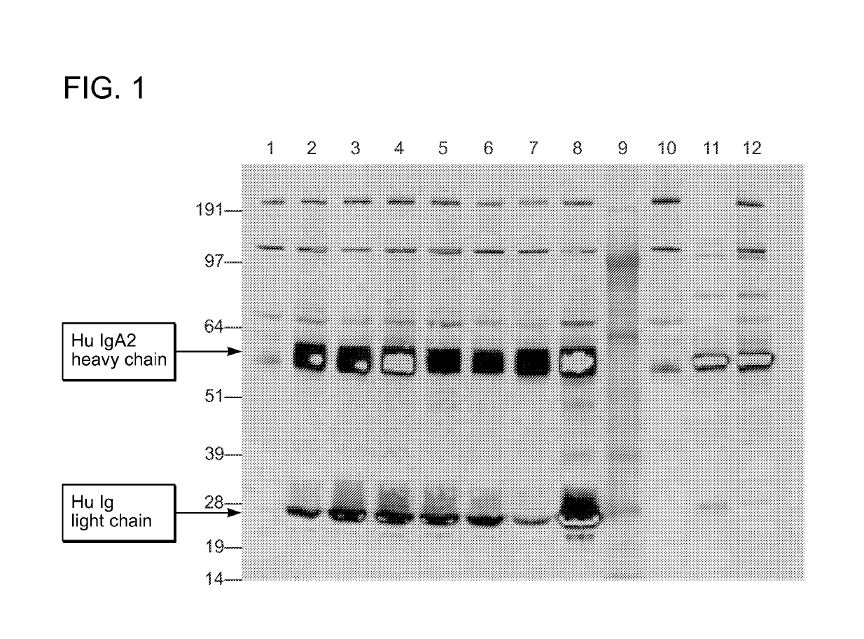

Milk specific expression vectors were constructed by directly cloning fragments encoding the b12 light chain and b12 A2 heavy chain into the GTC goat β-casein expression vector. This vector carries the promoter and downstream untranslated region of goat β-casein (pBC1, Invitrogen). It directs expression of linked genes to the mammary gland with subsequent secretion into the milk. The resulting plasmids, BC2470HC and BC2469LC with heavy or light chain linked to the β-casein promoter, were micro-injected into pre-implantation mouse embryos and implanted into foster mothers. The resulting progeny were screened for the presence of the transgenes in their DNA. The founder female mice that carried the integrated beta casein linked to the heavy and light chains of the antibody were grown to maturity and bred. Founder males were bred to wild-type mice and the F1 progeny tested for the presence of the transgene. Transgenic founder and F1 females were then bred and their milk tested for the presence of the b12 IgA antibody. Subsequent generations of these mice were expanded and their ability of the transgenic females to produce the b12 IgA was confirmed (data not shown). Lines were identified that produced more than 5 mg/ml as judged by Western blot analysis (FIG. 1). The milk was pooled from a number of lines and put through a clarification step to remove the colloidal milk proteins. The resulting clarified milk containing the b12 IgA2 antibody was then used for antigen binding and neutralization studies, as well as a source of purified b12 IgA2 antibody.

### Neutralization of HIV by Milk Expressed b12 IgA2

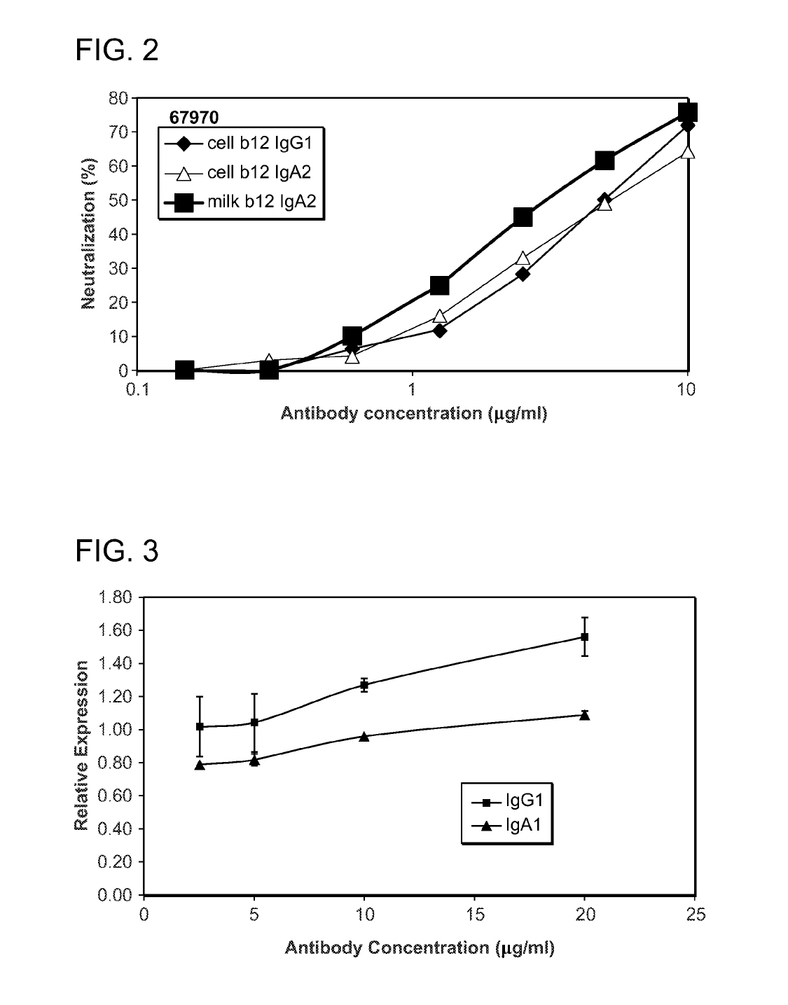

Both cell line derived and milk derived b12 IgA2 were tested for immunoreactivity with gp120 by ELISA. Parental IgG1 b12 was used as the control and a goat anti-κ reagent was used for detection which equivalently recognizes the common light chain for all constructs. Immunoreactivity was similar for both cell line derived and milk derived b12 IgA2 (data not shown). Neutralization of virus was determined using TZM-b1 cells with parental cell line derived IgG1 b12 used as a control. As shown in the representative experiments in FIG. 2, b12IgA2 in milk (square) was superior to cell derived IgA2 (triangle) and b12 IgG1 control (diamond) in neutralizing HIV (67970). To determine if this enhanced neutralization was a function of antibody alone or in concert with other milk proteins, b12IgA2 was purified from milk using protein L-chromatography. A comparison of neutralization capability of purified b12IgA2 derived from CHO cells, b12IgA2 purified from milk, and b12IgA2 remaining in the milk was performed. As shown in Table II, b12IgA2 expressed in milk was significantly more effective at neutralizing HIV. This synergistic effect was lost when purified away from other milk components.

TABLE II
Neutralization of HIV by Milk Derived b12 IgA2^{a}

| | CHO | | |
|---|---|---|---|
| | Purified^{b} | Milk Purified | Milk Expressed |
| SF162 (IC90)^{c} | 1.03 ± 0.52 | 0.79 ± 0.29 | 0.34 ± 0.13*^{d} |
| 67970 (IC50) | 8.68 ± 3.05 | 12.94 ± 5.47 | 1.52 ± 0.73* |
| JR-Fl (IC50) | 7.27 ± 2.45 | 27.60 ± 16.17 | 2.72 ± 1.49* |
| 89.6 (IC90) | 6.75 ± 2.63 | 8.37 ± 3.10 | 1.85 ± 1.53* |

^{a}Neutralization was measured using TZM-bl assay
^{b}b12 IgA2 antibody was purified from CHO cells, milk and tested as expressed in milk.
^{c}Virus isolate tested with indication of whether IC90 or IC50 values were used as determined by linear regression analysis
^{d}p < 0.05

### Antibody Dependent Cell-Mediated Viral Inhibition by Milk Expressed b12 IgA2

In addition to direct viral neutralization, antibody may also direct cell mediated inhibition of HIV as measured by ADCVI¹³. Neutrophils or PMN, which express IgA receptor were used in this assay as described previously. Serial dilutions of antibody were tested in the presence or absence of PMN to identify the contribution of direct neutralization to the inhibitory effect. As shown in Table III, b12IgA2 was not as effective at neutralizing virus as measured using the TZM-b1 assay. In fact, there was lack of neutralization for the majority of antibody/virus combinations when tested without PMN. However, in the presence of PMN, there was significant inhibitory activity with b12IgA2 in milk and consistently more effective than b12IgA2 purified from either CHO cells or milk. Thus, regardless of whether measuring neutralization of a single round of infection (TZM-b1) or cell-to-cell spread of HIV (ADCVI), b12IgA2 expressed in milk is significantly more effective at HIV inhibition.

TABLE III
ADCVI activity of b12 IgA2

| | Antibody Concentration for IC50 (μg/ml) | | | | | | | | | |
|---|---|---|---|---|---|---|---|---|---|---|
| | BaL | | SF162 | | 93MW960 | | 89.6 | | JR-FL | |
| | − | + | − | + | − | + | − | + | − | + |
| Antibody | PMN | PMN | PMN | PMN | PMN | PMN | PMN | PMN | PMN | PMN |
| b12 IgA2 | >20 | 2.1 | 9.5 | 0.3 | >20 | 10.0 | >20 | 14.5 | >20 | >20 |
| produced in | | | | | | | | | | |
| CHO | | | | | | | | | | |
| b12 IgA2 | 13.1 | 2.2 | 2.1 | 0.3 | >20 | 17.5 | >20 | 17.1 | >20 | >20 |
| purified from | | | | | | | | | | |
| milk | | | | | | | | | | |
| b12 IgA2 | 19.3 | 2.3 | 6.6 | 0.4 | >20 | 3.3 | 4.8 | 3.9 | 9.6 | 6.3 |
| produced in | | | | | | | | | | |
| milk | | | | | | | | | | |

### Example 2

### Monoclonal Antibodies, Virus, and Cell Lines

The neutralizing IgG antibody F425-A1g8 was generated as previously described (Cavacini et al., AIDS. 17:685-689, 2003), and was shown to bind to the CD4i site of gp 120. The immunoglobulin expression vectors pLC-HuCκ, pHC-HuCγ1, and pHC-HuCα1 were obtained which contained the human immunoglobulin light chain, heavy chain γ1 and alphal constant regions, respectively. The CHO-K1 cells were from American Type Culture Collection. The following reagents were obtained through the AIDS Research and Reference Reagent Program, Division of AIDS, NIAID, NIH: SF162 (R5) from Dr. Jay Levy; 89.6 (R5X4) from Dr. Ronald Collman; BaL (R5) from Dr. Suzanne Gartner, Dr. Mikulas Popovic, and Dr. Robert Gallo; 93MW960 (clade C, R5) from Dr. Robert Bollinger and the UNAIDS Network for HIV;

JR-FL (R5) from Dr. Irvin Chen; Isolate 67970 (CXCR4) was from Dr. David Montefiori. TZM-b1 cells from Dr. John C. Kappes, Dr. Xiaoyun Wu, and Transzyme, Inc.

### Construction, Production, and Purification of IgA F425A1g8 Antibody Variants

F425-A1g8 VH and VL were PCR amplified from a F425-A1 g8 hybridoma cell line using specific primers (Table 4) which introduced restriction enzymes sites (5′ NheI and 3′ HindIII for VH; 5′ NheI and 3′ NotI for VL). The VH fragment was cloned separately into the expression vectors pHC-HuCγ1 and pHC-huCα1. The VL was cloned into vector pLC-huCκ. Paired purified plasmids encoding the F425-A1g8 light chain versus IgG1 heavy chain and F425-A1g8 light chain versus IgA1 heavy chain were co-transfected into CHO-K1 cells in equimolar amounts in 6-well plates using lipofectamine LTX reagent (Invitrogen Life Technologies). Selection with G418 (800 μg/ml) and puromycin (10 mg/ml) were added after 24 hours. Cells were plated in 96-well plates with selection, and wells were screened when dense using standard IgG and IgA capture ELISAs. Positive wells were cloned by limiting dilution until a stable producing cell line was isolated. Antibody was purified from culture supernatant using protein L chromatography. Purity was confirmed using SDS-PAGE.

TABLE 4
Primers for amplifying variable domain of
F425-A1g8 and human J chain fragment

| | |
|---|---|
| F425A1g8VH-5′ | CTAGCTAGCCGCCACCATGGAGCTTGG |
| (Nhe I) | (SEQ ID NO: 3) |
| F425A1g8VH-3′ | CCCTTGAAGCTTGCTGAAGAGACGGTG |
| (Hind III) | (SEQ ID NO: 4) |
| F425A1g8VL-5′ | CTAGCTAGCCGCCACCATGGACATGAGG |
| (Nhe I) | (SEQ ID NO: 5) |
| F425A1g8VL-3′ | GACAGATGGTGCGGCCGCAGTTCGITTGATA |
| (Not I) | TCC (SEQ ID NO: 6) |
| Human J Chain-5′ | CTAGCTAGCATGAAGAACCATTTGC |
| (Nhe I) | (SEQ ID NO: 7) |
| Human J Chain-3′ | TGCGATATCTTAGTCAGGATAGCAGG |
| (EcoR V) | (SEQ ID NO: 8) |

Restriction sites are bolded.
VH: variable domain of heavy chain;
VL: variable domain of light chain;
J: human J chain.

### Immunoreactivity of Recombinant IgA F425A1g8 Antibody Variants

Live cell ELISA assay was performed to determine the immunoreactivity of F425-A1 g8 variances to the CD4 binding site. SF2 infected cells (1×10⁶) were incubated with antibody at 20, 10, 5, and 2.5 μg/ml for 30 minutes followed by washing and incubation with HRP-conjugated goat anti-human IgG or IgA (Southern Biotechnology Associates). The human monoclonal antibodies b12 IgG1 or IgA1 were run at 20 μg/ml as a standard to determine relative reactivity of the IgA F425-A1g8 antibody variants with HIV. After washing, cells were resuspended in 100 μl TMB substrate and incubated for 10 minutes. Reaction was stopped by adding 100 μl of 1M phosphoric acid and samples were read on a plate reader at 450 nm.

### Direct Viral Neutralization

The neutralization activity of isolated IgA F425-A1g8 antibody variants were determined in vitro using a TZM-b1 assay with a panel of three isolates including SF162, JR-FL, and 67970. Primary isolate virus was grown in PHA-stimulated peripheral blood mononuclear cells (PBMC) as previously described (Cavacini et al., AIDS Res. Hum. Retroviruses. 19:785-792; Cavacini et al., AIDS. 17:685-689, 2003; Wei et al., Antimicrob. Agents Chemother. 46:1896-1905, 2002) and titered on TZM-b1 cells (Duval et al., J. Virol. 82:4671-4674, 2008) to determine TCID₅₀. Serial two-fold dilutions of IgA F425-A1g8 antibody variants were incubated with virus stock diluted to 100 TCID₅₀ for 1 hour at 37° C. prior to the addition of TZM-b1 cells (1×10⁴ c/well).

Using β-galactosidase reagent from Promega as an indicator of HIV replication, plates were incubated for 48 hours at 37° C. and 5% CO₂ prior to the measurement of β-galactosidase activity.
Percent neutralization was determined based on control wells of virus and media and IC₅₀ and IC₉₀ values calculated by regression curve analysis.

### Antibody Dependent Cell-Mediated Viral Inhibition (ADCVI)

ADCV1 activity was measured using HIV grown in PHA-stimulated PBMC as previously described (Miranda et al, J. Immunol. 178:7132-7138, 2007). Neutrophils were obtained from peripheral blood of sero-negative donors by Ficoll-Hypaque gradient centrifugation. Antibodies were titered in 96-well, round-bottom plates in 50 μl of media containing 20% heat-inactivated FBS. Target cells were PBMC productively infected with HIV-1 four days prior to use as previously described (Cavacini et al., J. Virol. 73:9638-9641, 1999), and 1×10⁵ infected cells were added per well in 50 μl Within 10 minutes of the combination of antibody and infected cells, neutrophils were added to the wells at 1×10⁶ effector cells/well in 100 μl, resulting in a effector:target (E:T) ratio of 10:1. After 4 hours, in order to measure the surviving infectious virus, PHA stimulated PBMC were added as indicator cells (1×10⁵/well). These indicator PBMC were incubated for seven days in the presence of IL-2 at which time the supernatant was quantitated for p24 by a p24-specific ELISA (Stubbe et al., J. Immunol. 164:1952-1960, 2000). IC₅₀ values were determined by linear regression analysis and significance was ascertained by student's t-test. Control wells included irrelevant antibody, no effectors, or no targets to determine background release of virus, maximal production of virus, and whether PMN alone were infected, respectively. Viral inhibition was calculated based on the p24 amount from an irrelevant antibody control. Experiments were repeated three to five times.

### Example 3

### Immunoreactivity of F425-A1g8 IgA Antibody Variants

To determine the immunoreactivity of F425-A1g8 antibody variants with the CD4i epitope on HIV infected cells, a live cell ELISA assay was used. Since HRP-conjugated secondary antibodies directly binding to the light chain may be competed by antigen, IgG or IgA isotype-specific secondary antibodies had to be used. Therefore, b12 IgG1 and IgA1 were used to establish relative reactivity by comparing the absorbance (optical density) obtained with F425-A1g8 antibody variants with that obtained from the b12 controls. The results are expressed as a “relative expression” b12 unit (OD F425-A1g8/OD b12). As shown in FIG. 3, the reactivity of F425-A1g8 IgA1 with HIV was retained. In this experiment, SF2-infected cells (1×10⁶) were incubated with titered antibodies of F425-A1g8 IgG1 (square) and IgA1 (triangle) which were detected using HRP-conjugated goat anti-human IgG or IgA. Bound antibody was visualized using TMB substrate and stopped by 100 μl of 1M phosphoric acid. The OD was read on plate reader at 450 nm. b12 IgG1 or IgA1 (20 microg/ml) was used as a standard to determine relative reactivity of the F425-A1g8 variants with HIV. In particular, the IgG1 variant of F425-A1g8 had more relative binding than that observed for the IgA1 variant.

### Example 4

### Neutralizing Activity of F425-A1g8 IgA Antibody Variants Against HIV-1

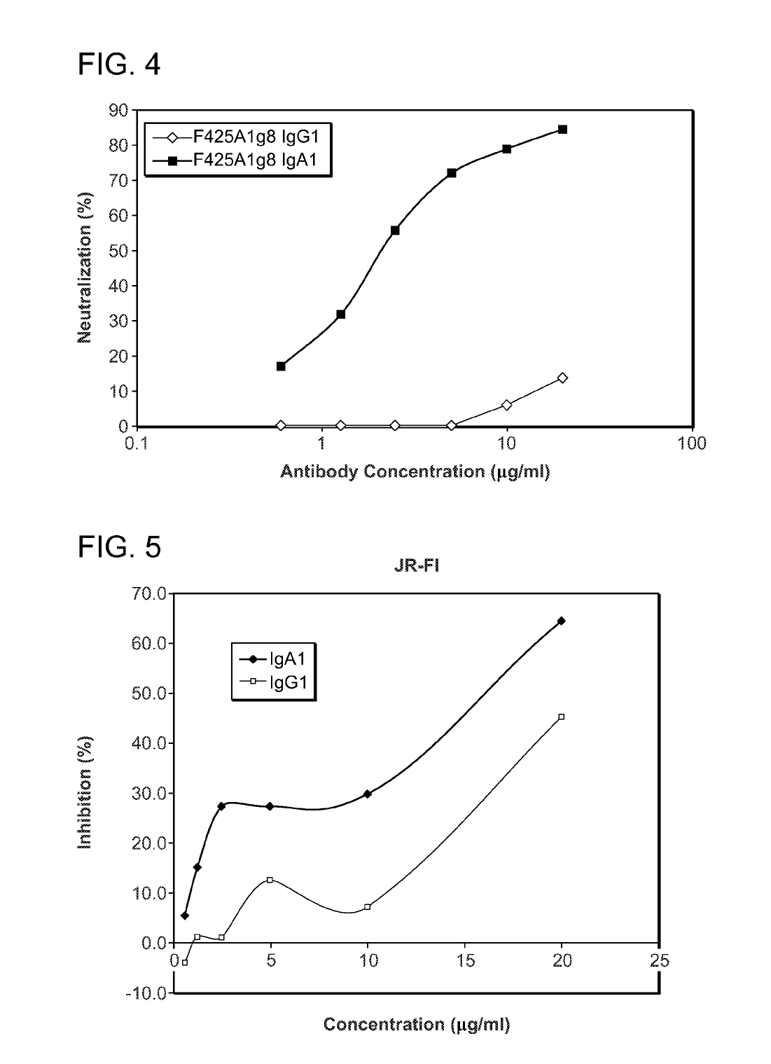

Neutralization of HIV was tested using TZM-b1 cells and three clade B primary isolate viruses (SF162, JR-FL, 67970) grown in PBMCs. Serial dilutions of antibody were tested and IC₅₀ values for JR-FL and 67970, and IC₉₀ for SF162 were determined by linear regression. In contrast to minimal neutralization by F425-A1g8 IgG1 in the absence of soluble CD4 (sCD4), the IgA1 variant of the antibody displayed significant neutralization activity against a number of HIV clade B isolates in the absence of sCD4 as shown in Table 5 and FIG. 4. As shown in FIG. 4, the neutralizing activity against JR-FL by the IgA1 antibody variant of F425-A1g8 was significantly increased compared to that of the F425-A1 g8 IgG1 antibody. In this experiment, JR-FL (100 TCID₅₀) was incubated with two-fold serial dilutions of F425-A1g8 IgG1 (open diamonds) and IgA I (black squares) antibody variants for one hour prior to the addition of TZM-b1 cells. HIV was measured as β-galactosidase activity after 48 hours. Percent neutralization was determined by the formula ((control−test)/control)×100.

As shown in Table 5, even though the F425-A1 g8 IgG1 antibody neutralized the SF162 isolate, the IgA1 antibody variant of F425-A1g8 displayed significantly increased neutralization activity. These results were the mean of triplicate wells and were representative of at least three independent experiments. This differential neutralization was confirmed in studies using tier 1

and reference panel virus (n=7 including BaL and SF162) grown in 293T cells. Increased neutralization mediated by IgA1 occurs despite relatively decreased immunoreactivity of the IgA1 to SF2 infected cells as compared to the IgG1.

TABLE 5
Neutralization of HIV-1 by F425-A1g8
IgG and IgA antibody variants

| | JR-FL | 67970 | SF162 |
|---|---|---|---|
| | Clade B, R5 | Clade B, X4 | Clade B, R5 |
| | (IC₅₀)^{a} | (IC₅₀) | (IC₉₀)^{b} |
| IgG1^{c} | >40 | >40 | 2.3 ± 1.4 |
| IgA1^{d} | 1.73 ± 0.2 | 23.3 ± 14.3 | 1.7 ± 1.0 |

^{a} and ^{b}IC₅₀ or IC₉₀ concentration (μg/m1) of antibody required for 50% or 90% inhibition of HIV, respectively.
^{c}F425-A1g8 IgG1 antibody variant expressed from CHO-K1 cells.
^{d}F425-A1g8 IgAl antibody variant expressed from CHO-K1 cells.

### Example 5

### Functional Activity of F425-A1g8 IgA Antibody Variant in Mediating ADCVI

The impact of the IgA1 constant domain of the F425-A1g8 IgA antibody variant on functional ability of ADCVI for HIV and HIV-infected cells was investigated. HIV-1-binding antibodies mediate ADCVI through an interaction with specific Fe receptors on effector cells, resulting in effector cell-mediated destruction of infected cells with antibody-bound antigen (Forthal et al., J. Immunol. 178:6596-6603, 2007). Therefore, ADCVI would be a useful assay to determine the ability of the isotype variants of specific antibodies to mediate effector cell destruction of or inhibit HIV replication in an infected target cell population in vivo. Polymorphonuclear leukocytes (PMN) or neutrophils are the predominant (60-70%) type of white blood cell in the circulation and play a critical role in innate immunity against infections. PMN consistently express multiple receptors for IgG including FcγRIIa (CD32), FcγRIIIa (CD16), and FcγRIIIb. They also express FcγRI (CD64) following induction with G-CSF. In addition to Fc receptors for IgG, PMN also express Fc receptors for IgA (FcαR, CD89). Crosslinking Fcgamma receptors as well as cross-linking of the IgA receptor on PMN by monoclonal antibodies have been shown to be critical to induce ADCC against tumor cells (Hernandez-Ilizaliturri et al., Clin. Cancer Res. 9:5866-5873, 2003; Rafiq et al., J. Clin. Invest. 110:71-79, 2002). Therefore, although traditional ADCVI (or ADCC) assays are based on mononuclear cell populations, we propose to use neutrophils as effectors.

Since the binding of F425-A1g8 was different with strains of virions, a total of five isolates, including clade B representing R5, R5X4, and X4 isolates and clade C isolate (R5), were tested in this variant of the neutralization assay. Antibody-mediated destruction of HIV and HIV-infected cells is determined by testing the inhibition of subsequent HIV replication or p24 levels. The results of these assays are summarized in Table 6 as well as in FIG. 5, which depicts data specifically of the tested JR-FL strain. As exemplified in FIG. 5, the F425-A1g8 IgA1 antibody variant showed more robust ADCVI activity for clade B isolate JR-FL compared to that of the IgG1 antibody variant. For these experiments, F425-A1g8 variants were incubated with virus-infected (e.g., JR-FL infected) PBMC just prior to adding neutrophils at an E:T ratio of 10:1. After 4 hours, PHA stimulated PBMC were added as indicator cells and p24 was quantitated by ELISA after one week. Percent inhibition was determined by the formula: [(p24 control-p24 test)/p24 control]×100.

As shown in Table 6, the F425-A1g8 IgA1 antibody variant showed significant ADCV1 activity for both clade B isolates and a single clade C isolate. For two of four clade B isolates (SF162 and JR-FL, both R5), F425A1g8 IgG1 failed to mediate ADCVI activity whereas significant activity was observed for the F425-A1g8 A1 antibody variant with p-values ranging from 0.0008-0.05 for multiple experiments. Two clade B strains, BaL (R5) and 89.6 (R5X4) failed to be inhibited by either isotype variant at the concentrations tested. Both antibody isotype variants inhibited the clade C isolate, 93MW960. The IgG1 isotype had greater activity against the Clade C isolate than IgA1 (p-value from 0.0012 to 0.0598).

The variant in impact of isotype in ADCVI may result from affinity and/or binding specificity of the Fc fragment of the IgA1 subclass (compared to the IgG1 subclass) with Fc receptors on the surface of neutrophils. On the other hand, the antigen density and epitope orientation may result in differences in outcome. There was no viral inhibition in mock control wells which contained antibody, target cells, or indicator cells without neutrophils. Viral replication was similar for control wells containing effector cells, target cells without antibody, and target cells alone.

TABLE 6
ADCVI activity of HIV-1 by F425A1g8
IgAl and IgG1 antibody variants

| | IC₅₀(μg/m1)^{a} | | | | |
|---|---|---|---|---|---|
| | BaL | JR-FL | 93MW960 | 89.6 | SF162 |
| | Clade | Clade | Clade | Clade | Clade |
| | B, R5 | B, R5 | C R5 | B, R5X4 | B, R5 |
| | (n = 6) | (n = 6) | (n = 5) | (n = 3) | (n = 3) |
| IgG1 | >40 | >40 | 9.5 ± 7.9 | >40 | >40 |
| IgAl | >40 | 16.6 ± 5.1 | 18.3 ± 13.4 | >40 | 6.1 ± 5.9 |

^{a}The ADCVI activity was detennined by IC₅₀ that represents concentration (mg/ml) of antibody required for 50% inhibition of HIV.

### Example 6

### Sequences of Antibodies

### 1. F425-A1g8 Antibody

A. F425-A1g8 VH+IgA1 constant domain
a. F425-A1g8 Heavy chain variable domain:

(SEQ ID NO: 9)
QEQLVESGGGVVQPGRSLRLSCEASGFIFSAFVLHWVRQAPGKGLEWVA
VVWYDGNSKYYADSVKGRFTISRDTSQNTLHLQMDSLRPEDTAVYYCAR
EWVADDDTFDGFDVWGQGTMVTVSS
(SEQ ID NO: 10)
CAGGAGCAGCTGGTGGAGTCTGGGGGAGGAGTGGTCCAGCCTGGGAGGT
CCCTGAGACTCTCCTGTGAAGCGTCTGGTTTCATCTTCAGTGCCTTTGT
CTTGCACTGGGTCCGACAGGCTCCAGGCAAGGGGCTGGAGTGGGTGGCA
GTTGTTTGGTATGATGGAAATAGTAAATACTATGCAGACTCCGTGAAGG
GCCGATTCACCATCTCCAGAGACACTTCCCAGAACACACTGCATCTGCA
AATGGACAGCCTGCGTCCCGAGGACACGGCTGTCTATTACTGTGCGAGA
GAATGGGTGGCGGACGATGATACTTTTGATGGTTTTGATGTCTGGGGCC
AAGGGACAATGGTCACCGTCTCTTCA

b. IgA1 heavy chain constant domain:

(SEQ ID NO: 11)
ASLTSPKVFPLSLCSTQPDGNVVIACLVQGFFPQEPLSVTWSESGQGVTA
RNFPPSQDASGDLYTTSSQLTLPATQCLAGKSVTCHVKHYTNPSQDVTVP
CPVPSTPPTPSPSTPPTPSPSCCHPRLSLHRPALEDLLLGSEANLTCTLT
GLRDASGVTFTWTPSSGKSAVQGPPDRDLCGCYSVSSVLSGCAEPWNHGK
TFTCTAAYPESKTPLTATLSKSGNTFRPEVHLLPPPSEELALNELVTLTC
LARGFSPKDVLVRWLQGSQELPREKYLTWASRQEPSQGTTTFAVTSILRV
AAEDWKKGDTFSCMVGHEALPLAFTQKTIDRLAGKPTHVNVSVVMAEVDG
TCY
(SEQ ID NO: 12)
gcaagcttgaccagccccaaggtcttcccgctgagcctctgcagcaccca
gccagatgggaacgtggtcatcgcctgcctggtccagggcttcttccccc
aggagccactcagtgtgacctggagcgaaagcggacagggcgtgaccgcc
agaaacttcccacccagccaggatgcctccggggacctgtacaccacgag
cagccagctgaccctgccggccacacagtgcctagccggcaagtccgtga
catgccacgtgaagcactacacgaatcccagccaggatgtgactgtgccc
tgcccagttccctcaactccacctaccccatctccctcaactccacctac
cccatctccctcatgctgccacccccgactgtcactgcaccgaccggccc
tcgaggacctgctcttaggttcagaagcgaacctcacgtgcacactgacc
ggcctgagagatgcctcaggtgtcaccttcacctggacgccctcaagtgg
gaagagcgctgttcaaggaccacctgaccgtgacctctgtggctgctaca
gcgtgtccagtgtcctgtcgggctgtgccgagccatggaaccatgggaag
accttcacttgcactgctgcctaccccgagtccaagaccccgctaaccgc
caccctctcaaaatccggaaacacattccggcccgaggtccacctgctgc
cgccgccgtcggaggagctggccctgaacgagctggtgacgctgacgtgc
ctggcacgtggcttcagccccaaggatgtgctggttcgctggctgcaggg
gtcacaggagctgccccgcgagaagtacctgacttgggcatcccggcagg
agcccagccagggcaccaccaccttcgctgtgaccagcatactgcgcgtg
gcagccgaggactggaagaagggggacaccttctcctgcatggtgggcca
cgaggccctgccgctggccttcacacagaagaccatcgaccgcttggcgg
gtaaacccacccatgtcaatgtgtctgttgtcatggcggaggtggacggc
acctgctac

B. F425-A1g8 VL+Light Chain constant domain
a. F425-A1g8 Light chain variable domain:

(SEQ ID NO: 13)
EIVLSQSPATLSLSPGERATLSCRASQSVTNSLAWYQQKPGQAPRLLIYD
ASNRATGIPARFSGSGSGTDFTLTISSLEPEDFAVYYCQQRSNWPPEVTF
GPGTKVDIKR
(SEQ ID NO: 14)
GAAATTGTGTTGTCACAGTCTCCAGCCACCCTGTCTTTGTCTCCAGGGGA
AAGAGCCACCCTCTCCTGCAGGGCCAGTCAGAGTGTTACCAACTCCTTAG
CCTGGTACCAACAGAAACCTGGCCAGGCTCCCAGGCTCCTCATCTATGAT
GCATCCAACAGGGCCACTGGCATCCCAGCCAGGTTCAGTGGCAGTGGGTC
TGGGACAGACTTCACTCTCACCATCAGCAGCCTAGAGCCTGAAGATTTTG
CAGTTTATTACTGTCAGCAGCGTAGCAACTGGCCTCCGGAGGTCACTTTC
GGCCCTGGGACCAAAGTGGATATCAAACGA

b. IgA1 light chain constant domain:

(SEQ ID NO: 15)
TAAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC
(SEQ ID NO: 16)
actgcggccgcaccatctgtcttcatcttcccgccatctgatgagcagtt
gaaatctggaactgcctctgttgtgtgcctgctgaataacttctatccca
gagaggccaaagtacagtggaaggtggataacgccctccaatcgggtaac
tcccaggagagtgtcacagagcaggacagcaaggacagcacctacagcct
cagcagcaccctgacgctgagcaaagcagactacgagaaacacaaagtct
acgcctgcgaagtcacccatcagggcctgagctcgcccgtcacaaagagc
ttcaacaggggagagtgt

C. CDR sequences of F425-A1g8 Heavy chain

a.
CDR-H1:
(SEQ ID NO: 17)
GFIFSAFV
CDR-H2:
(SEQ ID NO: 18)
VWYDGNSK
CDR-H3:
(SEQ ID NO: 19)
AREWVADDDTFDGFDV
b.
CDR-H1:
(SEQ ID NO: 20)
GGTTTCATCTTCAGTGCCTTTGTC
CDR-H2:
(SEQ ID NO: 21)
GTTTGGTATGATGGAAATAGTAAA
CDR-H3:
(SEQ ID NO: 22)
GCGAGAGAATGGGTGGCGGACGATGATACTTTTGATGGTTTTGATGTC

D. CDR sequences of F425-A1g8 Light chain

a.
CDR-L1:
(SEQ ID NO: 23)
QSVTNS
CDR-L2
(SEQ ID NO: 24)
DAS
CDR-L3
(SEQ ID NO: 25)
QQRSNWPPEVT
b.
CDR-L1:
(SEQ ID NO: 26)
CAGAGTGTTACCAACTCC
CDR-L2:
(SEQ ID NO: 27)
GATGCATCC
CDR-L3:
(SEQ ID NO: 28)
CAGCAGCGTAGCAACTGGCCTCCGGAGGTCACT

B. b12 IgA2 antibody

Heavy chain
(SEQ ID NO: 29)
M E F G L S W V F L V A I I I G V Q C Q V Q L V Q
S G A E V K K P G A S V K V S C Q A S G Y R F S N
F V I H W V R Q A P G Q R F E W M G W I N P Y N G
N K E F S A K F Q D R V T F T A D T S A N T A Y M
E L R S L R S A D T A V Y Y C A R V G P Y S W D D
S P Q D N Y Y M D V W G Q G T T V I V S S A S L T
S P K V F P L S L D S T P Q D G N V V V A C L V Q
G F F P Q E P L S V T W S E S G Q N V T A R N F P
P S Q D A S G D L Y T T S S Q L T L P A T Q C P D
G K S V T C H V K H Y T N S S Q D V T V P C R V P
P P P P C C H P R L S L H R P A L E D L L L G S E
A N L T C T L T G L R D A S G A T F T W T P S S G
K S A V Q G P P E R D L C G C Y S V S S V L P G C
A Q P W N H G E T F T C T A A H P E L K T P L T A
N I T K S G N T F R P E V H L L P P P S E E L A L
N E L V T L T C L A R G F S P K D V L V R W L Q G
S Q E L P R E K Y L T W A S R Q E P S Q G T T T Y
A V T S I L R V A A E D W K K G E T F S C M V G H
E A L P L A F T Q K T I D R L A G K P T H I N V S
V V M A E A D G T C Y
Light chain
(SEQ ID NO: 30)
M E F G L S W V F L V A I I I G V Q C Q V E I V L
T Q A P G T L S L S P G E R A T F S C R S S H S I
R S R R V A W Y Q H K P G Q A P R L V I H G V S N
R A S G I S D R F S G S G S G T D F T L T I T R V
E P E D F A L Y Y C Q V Y G A S S Y T F G Q G T K
L E R K R T A A A P S V F I F P P S D E Q L K S G
T A S V V C L L N N F Y P R E A K V Q W K V D N A
L Q S G N S Q E S V T E Q D S K D S T Y S L S S T
L T L S K A D Y E K H K V Y A C E V T H Q G L S S
P V T K S F N R G E C

### REFERENCES

1. Becquart, P. et al. Compartmentalization of the IgG immune response to HIV-1 in breast milk. Aids 13, 1323-1331 (1999).
2. Duprat, C. et al. Human immunodeficiency virus type 1 IgA antibody in breast milk and serum. Pediatr Infect Dis J 13, 603-608 (1994).
3. Becquart, P. et al. Secretory anti-human immunodeficiency virus (HIV) antibodies in colostrum and breast milk are not a major determinant of the protection of early postnatal transmission of HIV. J Infect Dis 181, 532-539 (2000).
4. Van de Perre, P. et al. Infective and anti-infective properties of breastmilk from HIV-1-infected women. Lancet 341, 914-918 (1993).
5. Mantis, N. J. et al. Inhibition of HIV-1 infectivity and epithelial cell transfer by human monoclonal IgG and IgA antibodies carrying the b12 V region. J Immunol 179, 3144-3152 (2007).
6. Roben, P. et al. Recognition properties of a panel of human recombinant Fab fragments to the CD4 binding site of gp 120 that show differing abilities to neutralize human immunodeficiency virus type 1. J Virol 68, 4821-4828 (1994).
7. Duval, M., Posner, M. R. & Cavacini, L. A. A bispecific antibody composed of a nonneutralizing antibody to the gp41 immunodominant region and an anti-CD89 antibody directs broad human immunodeficiency virus destruction by neutrophils. J Virol 82, 4671-4674 (2008).
8. Wei, X. et al. Emergence of resistant human immunodeficiency virus type 1 in patients receiving fusion inhibitor (T-20) monotherapy. Antimicrob Agents Chemother 46, 1896-1905 (2002).
9. Miranda, L. et al. The neutralization properties of a HIV-specific antibody are markedly altered by glycosylation events outside the antigen-binding domain. J Immunol 178, 7132-7138 (2007).
10. Huang, M. T. & Gorman, C. M. Intervening sequences increase efficiency of RNA 3′ processing and accumulation of cytoplasmic RNA. Nucleic Acids Res 18, 937-947 (1990).
11. Krugmann, S., Pleass, R. J., Atkin, J. D. & Woof, J. M. Structural requirements for assembly of dimeric IgA probed by site-directed mutagenesis of J chain and a cysteine residue of the alpha-chain CH2 domain. J Immunol 159, 244-249 (1997).
12. Sorensen, V., Rasmussen, I. B., Sundvold, V., Michaelsen, T. E. & Sandlie, I. Structural requirements for incorporation of J chain into human IgM and IgA. Int Immunol 12, 19-27 (2000).
13. Forthal, D., Gilbert, P., Landucci, G. & Phan, T. Recombinant gp120 vaccine-induced antibodies inhibit clinical strains of HIV-1 in the presence of Fc receptor-bearing effector cells and correlate inversely with HIV infection rate. J Immunol 178, 6596-6603 (2007).
14. Mascola, J. R. et al. Potent and synergistic neutralization of human immunodeficiency virus (HIV) type 1 primary isolates by hyperimmune anti-HIV immunoglobulin combined with monoclonal antibodies 2F5 and 2G12. J Virol 71, 7198-7206 (1997).
15. Mascola, J. et al. Protection of macaques against pathogenic simian/human immunodeficiency virus 89.6PD by passive transfer of neutralizing antibodies. J Virol 73, 4009-40018 (1999).
16. Mascola, J. R. et al. Protection of macaques against vaginal transmission of a pathogenic HIV-1/SIV chimeric virus by passive infusion of neutralizing antibodies. Nat Med 6, 207-210 (2000).
17. Ferrantelli, F. et al. Post-exposure prophylaxis with human monoclonal antibodies prevented SHIV89.6P infection or disease in neonatal macaques. AIDS 17, 201-209 (2003).
18. Baba, T. et al. Human neutralizing monoclonal antibodies of the IgG1 subtype protect against mucosal simian-human immunodeficiency virus infection. Nat Med 6, 200-206 (2000).
19. Ferrantelli, F. et al. Complete protection of neonatal rhesus macaques against oral exposure to pathogenic simian-human immunodeficiency virus by human anti-HIV monoclonal antibodies. J Infect Dis 189, 2167-2173 (2004).
20. Hofmann-Lehmann R et al. Postnatal pre- and postexposure passive immunization strategies: protection of neonatal macaques against oral simian-human immunodeficiency virus challenge. J Med Primatol 31, 109-119 (2002).
21. Hofmann-Lehmann, R. et al. Postnatal passive immunization of neonatal macaques with a triple combination of human monoclonal antibodies against oral simian-human immunodeficiency virus challenge. J Virol 75, 7470-7480 (2001).
22. Hoffmann-Lehmann R, R. R., Vlasak J, Smith B A, Baba T, Liska V, Montefiori D C, McClure H M, Anderson D C, Bernacky B J, Rizvi T A, Schmidt R, Hill L R, Keeling M E, Katinger H, Stiegler G, Posner M R, Cavacini L A, Chou T, Ruprecht R M. Passive immunization against oral AIDS virus transmission: An approach to prevent mother-to-infant transmission? Journal of Medical Primatology 30, 190-196 (2001).
23. Pilgrim, A. et al. Neutralizing antibody responses to human immunodeficiency virus type 1 in primary infection and long-term-nonprogressive infection. J Infect Dis 176, 924-932 (1997).
24. Zhou, J. Y. & Montefiori, D. C. Antibody-mediated neutralization of primary isolates of human immunodeficiency virus type 1 in peripheral blood mononuclear cells is not affected by the initial activation state of the cells. J Virol 71, 2512-2517 (1997).
25. Bailey, J. et al. Neutralizing antibodies do not mediate suppression of human immunodeficiency virus type 1 in elite suppressors or selection of plasma virus variants in patients on highly active anti-retroviral therapy. J Virol 80, 4758-4770 (2006).
26. Stubbe, H., Berdoz, J., Kraehenbuhl, J.-P. & Corthesy, B. Polymeric IgA is superior to monomeric IgA and IgG carrying the same variable domain in preventing Clostridium difficile toxin A damaging of T84 monolayers. J Immunol 164, 1952-1960 (2000).
27. Walsh, E. & Falsey, A. Humoral and mucosal immunity in protection from natural respiratory syncytial virus infection in adults. J Infect Dis 190, 373-378 (2004).
28. Rocha-Zavaleta, L., Barrios, t., Garcia-Carranca, A., Valdespino, V. & Cruz-Talonia, F. Cervical secretory immunoglobulin A to human papillomavirus type 16 (HPV-16) from HPV-16 infected women inhibit HPV16 virus-like particle-induced hemagglutination of mouse red blood cells. FEMS Immunol Med Microbiol 31, 47-51 (2001).
29. Feng, N. et al. Inhibition of rotavirus replication by a non-neutralizing, rotavirus VP6-specific IgA mAb. J Clin Invest 109, 1203-1213 (2002).

### EQUIVALENTS

The foregoing written specification is considered to be sufficient to enable one skilled in the art to practice the invention. The present invention is not to be limited in scope by examples provided, since the examples are intended as an illustration of certain aspects and embodiments of the invention. Other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method of treating HIV infection in a subject, the method comprising administering to the subject: a composition comprising IgA antibody; a composition comprising multimeric antibody; or a composition comprising highly glycosylated antibody, to treat HIV infection.

2.-5. (canceled)

6. The method of claim 1, wherein the IgA antibody is produced in the mammary gland of a transgenic non-human mammal.

7. (canceled)

8. A method of decreasing the chance of HIV infection in a subject, the method comprising administering to the subject: a composition comprising IgA antibody; a composition comprising multimeric antibody; or a composition comprising highly glycosylated antibody, to decrease the chance of HIV infection.

9.-12. (canceled)

13. The method of claim 8, wherein the IgA antibody is produced in the mammary gland of a transgenic non-human mammal.

14. (canceled)

15. The method of claim 8, wherein the method comprises decreasing the chance of HIV infection in a subject that receives breast milk, the method comprising administering to breast milk: a composition comprising IgA antibody; a composition comprising multimeric antibody; or a composition comprising highly glycosylated antibody, to decrease the chance of HIV infection in a subject that receives the breast milk.

16.-19. (canceled)

20. The method of claim 15, wherein the milk-produced IgA antibody is produced in the mammary gland of a transgenic non-human mammal.

21. (canceled)

22. The method of claim 8, wherein the method comprises suppressing mother-to-child transmission of HIV.

23. The method of claim 22, wherein the method comprises applying: a composition comprising IgA antibody; a composition comprising multimeric antibody; or a composition comprising highly glycosylated antibody to the nipple of the mother prior to breast feeding to suppress mother-to-child transmission of HIV.

24. (canceled)

25. The method of claim 8, wherein the method comprises suppressing mother-to-child transmission of HIV by expressing IgA antibody in one or more cells of the mammary gland of the mother to suppress mother-to-child transmission of HIV.

26. (canceled)

27. The method of claim 22, wherein the mother is not a wet nurse.

28. (canceled)

29. (canceled)

30. The method of claim 22, wherein the IgA antibody is produced in the mammary gland of a transgenic non-human mammal.

31.-39. (canceled)

40. The method of claim 1, wherein the administration or application results in the suppression of HIV replication in a target cell population

41. The method of claim 1, wherein the composition is capable of inducing antibody-dependent cell-mediated viral inhibition (ADC VI).

42. The method of claim 1, further comprising administering anti-retroviral therapy

43. A composition comprising milk-produced IgA antibody.

44. The composition of claim 43, wherein the milk-produced IgA antibody is produced in the mammary gland of a transgenic non-human mammal.

45. (canceled)

46. (canceled)

47. The composition of claim 43, wherein the composition further comprises a pharmaceutically acceptable carrier.

48. The composition of claim 43, wherein the IgA antibody is an IgA1 antibody.

49. The composition of claim 43, wherein the IgA antibody is a dimeric IgA1 antibody.

50. The composition of claim 43, wherein the IgA antibody is an IgA2 antibody.

51.-62. (canceled)
